(19) United States
(12) Patent Application Publication
(10) Pub. No.: US 2009/0305978 A1
(43) Pub. Date: Dec. 10, 2009

(54) METHODS FOR INCREASING THE SIZE OF ANIMALS USING NEEDLELESS DELIVERY CONSTRUCTS

(75) Inventor: Doris Tham Zane, San Mateo, CA (US)

Correspondence Address: JONES DAY, 222 EAST 41ST ST, NEW YORK, NY, 10017, US

(73) Assignee: TRINITY BIOSYSTEMS, INC., Menlo Park, CA (US)

(21) Appl. No.: 12/282,853

(22) PCT Filed: Mar. 15, 2007
(86) PCT No.: PCT/US2007/006590

§ 371 Date: Apr. 20, 2009

Related U.S. Application Data
(60) Provisional application No. 60/783,534, filed on Mar. 16, 2006.

(51) Int. Cl. A61K 38/27; A61P 43/00

(52) U.S. Cl. 514/12

(57) ABSTRACT

The present invention relates, in part, to methods for increasing the size of a subject by administering a delivery construct comprising growth hormone to a subject. In one aspect, the method for increasing the size of a subject by at least about 12% comprises contacting an apical surface of a polarized epithelial cell of the subject with an amount of a delivery construct comprising growth hormone that is effective to increase the size of the subject by at least about 12%.

### 1. FIELD OF THE INVENTION

The present invention relates, in part, to methods for increasing the size of a subject by administering a delivery construct comprising a growth hormone to a subject. In one aspect, the method comprises contacting an apical surface of a polarized epithelial cell of the subject with an amount of a delivery construct comprising growth hormone that is effective to increase the size of the subject by at least about 12%.

### 2. BACKGROUND

Advances in biochemistry and molecular biology have resulted identification and characterization of many therapeutic macromolecules, including, for example, growth hormone (GH). Administration of GH can result in drastic improvements in quality of life for subjects afflict with a wide range of ailments.

However, administration of GH remains problematic. Currently, GH is typically administered by injection. Such injections require penetration of the subject's skin and tissues and are associated with pain. Further, penetration of the skin breaches one effective nonspecific mechanism of protection against infection, and thus can lead to potentially serious infection. In addition, injection of GH appears to be associated with induction of potentially adverse immune responses relative to other routes of administration.

Accordingly, efforts have been made to obtain methods and compositions that can be used to administer GH to subjects without breaching the skin of the subject. See U.S. application Ser. No. 11/244,349. However, additional methods and compositions are needed to optimize the therapeutic effects of such administration.

### 3. SUMMARY OF THE INVENTION

In certain aspects, the invention provides a method for increasing the size of a subject by at least about 12%, comprising contacting an apical surface of a polarized epithelial cell of the subject with an amount of a delivery construct effective to increase the size of the subject by at least about 12%, wherein said delivery construct comprises a receptor binding domain, a transcytosis domain, a cleavable linker, and growth hormone (GH), wherein the transcytosis domain transcytoses the GH to and through the basal-lateral membrane of said epithelial cell, and wherein cleavage at said cleavable linker separates said GH from the remainder of said construct, thereby delivering the GH to the subject in an amount effective to increase the size of the subject by at least about 12%.

In certain embodiments, the receptor binding domain is selected from the group consisting of receptor binding domains from Pseudomonas exotoxin A, cholera toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain binds to a cell surface receptor selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

In certain embodiments, the transcytosis domain is selected from the group consisting of transcytosis domains from Pseudomonas exotoxin A, botulinum toxin, diptheria toxin, pertussis toxin, cholera toxin, heat-labile E. coli enterotoxin, shiga toxin, and shiga-like toxin.

In certain embodiments, the cleavable linker is cleavable by an enzyme that is selected from the group consisting of Cathepsin GI, Chymotrypsin I, Elastase I, Subtilisin AI, Subtilisin AII, Thrombin I, and Urokinase I. In certain embodiments, the cleavable linker comprises an amino acid sequence that is selected from the group consisting of Ala-Ala-Pro-Phe (SEQ ID NO.:1), Oly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7).

In certain embodiments, the epithelial cell is selected from the group consisting of nasal epithelial cells, oral epithelial cells, intestinal epithelial cells, rectal epithelial cells, vaginal epithelial cells, and pulmonary epithelial cells. In certain embodiments, the epithelial cell is a nasal epithelial cell. In certain embodiments, the epithelial cell is an intestinal epithelial cell.

In certain embodiments, the subject is a mouse or rat. In certain embodiments, the subject is a human.

In certain embodiments, the delivery construct contacts the apical membrane of the epithelial cell.

In certain embodiments, the size of the subject is increased by at least about 13%. In certain embodiments, the size of said subject is increased by at least about 14%. In certain embodiments, the size of said subject is increased by at least about 15%. In certain embodiments, the size of said subject is increased by at least about 16%. In certain embodiments, the size of said subject is increased by at least about 17%. In certain embodiments, the size of said subject is increased by at least about 18%.

In certain embodiments, the size of said subject that is increased is a weight of said subject. In certain embodiments, the size of said subject that is increased is a height of said subject. In certain embodiments, the size of said subject that is increased is a length of said subject.

In certain embodiments, the GH is human growth hormone (hGH). In certain embodiments, the hGH has an amino acid sequence that is SEQ ID NO.:8.

In certain embodiments, the methods further comprise performing a method of the invention a second time about 1 day after the method of the invention is performed the first time. In certain embodiments, the methods further comprise performing a method of the invention a second time about 2 days after the method of the invention is performed the first time. In certain embodiments, the methods further comprise performing a method of the invention a second time about 3 days after the method of the invention is performed the first time.

### 4. BRIEF DESCRIPTION OF THE DRAWINGS

FIGS. 1A and 1B present the amino acid sequence of HGH Delivery Construct (SEQ ID NO:9), an exemplary Delivery Construct for delivering hGH.

FIG. 2 presents a graphical comparison of weight gain in lit/lit mice administered 30 μg hGH subcutaneously or intranasally with the HGH Delivery Construct.

FIG. 3 presents a graphical comparison of weight gain in lit/lit mice administered 60 μg hGH subcutaneously or intranasally with the HGH Delivery Construct.

FIG. 4 presents a graphical comparison of weight gain in lit/lit mice administered either 30 μg hGH or 60 μg hGH subcutaneously.

FIG. 5 presents a graphical comparison of weight gain in lit/lit mice administered either 30 μg hGH or 60 jig hGH intranasally with the HGH Delivery Construct.

FIG. 6 presents a table showing growth of lit/lit mice administered 30 μg hGH or 60 μg hGH subcutaneously or intranasally with the HGH Delivery Construct normalized to growth of mice not administered any hGH.

FIG. 7 presents a graphical representation of amounts of hGH observed in the serum of mice administered 30 μg hGH subcutaneously, 300 μg hGH orally with the HGH Delivery Construct, and 60 μg hGH intranasally with the HGH Delivery Construct.

FIG. 8 presents a graphical representation of amounts of bioactive hGH observed in the serum of mice administered 30 μg hGH subcutaneously, 300 μg hGH orally with the HGH Delivery Construct, and 60 μg hGH intranasally with the HGH Delivery Construct.

FIG. 9 presents a graphical representation of amounts of IGF-1 observed in the serum of mice administered 30 μg hGH subcutaneously, 300 μg hGH orally with the HGH Delivery Construct, and 60 μg hGH intranasally with the HGH Delivery Construct.

FIG. 10 presents a graphical representation of amounts of IGF1-BP3 observed in the serum of mice administered 30 μg hGH subcutaneously, 300 μg hGH orally with the HGH Delivery Construct, and 60 μg hGH intranasally with the HGH Delivery Construct.

FIG. 11 presents a graphical representation of amounts of anti-hGH IgG antibodies observed in the serum of mice administered 30 μg hGH subcutaneously, 300 μg hGH orally with the HGH Delivery Construct, and 60 μg hGH intranasally with the HGH Delivery Construct.

FIG. 12 presents a graphical representation of amounts of anti-ntPE IgG antibodies observed in the serum of mice administered 30 μg hGH subcutaneously, 300 μg hGH orally with the HGH Delivery Construct, and 60 μg hGH intranasally with the HGH Delivery Construct.

FIG. 13 presents a graphical representation of amounts of corticosterone observed in the serum of mice administered 30 μg hGH subcutaneously, 300 μg hGH orally with the HGH Delivery Construct, and 60 μg hGH intranasally with the HGH Delivery Construct.

FIG. 14 presents a graphical representation of amounts of leptin observed in the serum of mice administered 30 μg hGH subcutaneously, 300 μg hGH orally with the HGH Delivery Construct, and 60 μg hGH intranasally with the HGH Delivery Construct.

FIG. 15 presents a graphical representation of amounts of insulin observed in the serum of mice administered 30 μg hGH subcutaneously, 300 μg hGH orally with the HGH Delivery Construct, and 60 μg hGH intranasally with the HGH Delivery Construct.

FIG. 16 presents a table summarizing amounts of hGH, bioactive hGH, IGF-1, IGF1-BP3, anti-hGH IgG antibodies, anti-ntPE IgG antibodies, corticosterone, leptin, insulin, and ntPE observed in mouse serum 30 minutes following the day 10 administration of 30 μg hGH administered SC, 30 μg hGH administered orally with the HGH Delivery Construct, 30 μg hGH administered intranasally with the HGH delivery construct, 300 μg hGH administered orally with the HGH Delivery Construct, 60 μg hGH administered intranasally with the HGH delivery construct, and 60 μg hGH administered subcutaneously.

FIG. 17 presents a graphical representation of the pharmacokinetic profile of hGH serum concentration following administration of 30 μg hGH intranasally with the HGH Delivery Construct to BALB/c mice.

FIG. 18 presents a table showing amounts of hGH observed in the serum of BALB/c mice at various time points following intranasal administration.

### 5. DETAILED DESCRIPTION OF THE INVENTION

5.1. Definitions

Unless defined otherwise, all technical and scientific terms used herein have the meaning commonly understood by a person skilled in the art to which this invention belongs. As used herein, the following terms have the meanings ascribed to them unless specified otherwise.

A “ligand” is a compound that specifically binds to a target molecule. Exemplary ligands include, but are not limited to, an antibody, a cytokine, a substrate, a signaling molecule, and the like.

A “receptor” is compound that specifically binds to a ligand.

A ligand or a receptor (e.g., an antibody) “specifically binds to” or “is specifically immunoreactive with” another molecule when the ligand or receptor functions in a binding reaction that indicates the presence of the molecule in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to another polynucleotide comprising a complementary sequence and an antibody specifically binds under immunoassay conditions to an antigen bearing an epitope used to induce the antibody.

“Immunoassay” refers to a method of detecting an analyte in a sample involving contacting the sample with an antibody that specifically binds to the analyte and detecting binding between the antibody and the analyte. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity. In one example, an antibody that binds a particular antigen with an affinity (Kₘ) of about 10 μM specifically binds the antigen.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc. when there is such a change in environment following transcytosis of the delivery construct across a polarized epithelial membrane.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co., Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

Preferred pharmaceutical carriers depend upon the intended mode of administration of the active agent. Typical modes of administration include enteral (e.g., oral, intranasal, rectal, or vaginal) or parenteral (e.g., subcutaneous, intramuscular, intravenous or intraperitoneal injection; or topical (e.g., transderrnal, or transmucosal administration).

“Small organic molecule” refers to organic molecules of a size comparable to those organic molecules generally used in pharmaceuticals. The term excludes organic biopolymers (e.g., proteins, nucleic acids, etc.). Preferred small organic molecules range in size up to about 5000 Da, up to about 2000 Da, or up to about 1000 Da.

A “subject” of diagnosis, treatment, or administration is a human or non-human animal, including a mammal or a primate, and preferably a human.

“Treatment” refers to prophylactic treatment or therapeutic treatment. A “prophylactic ” treatment is a treatment administered to a subject who does not exhibit signs of a disease or exhibits only early signs for the purpose of decreasing the risk of developing pathology. A “therapeutic” treatment is a treatment administered to a subject who exhibits signs of pathology for the purpose of diminishing or eliminating those signs.

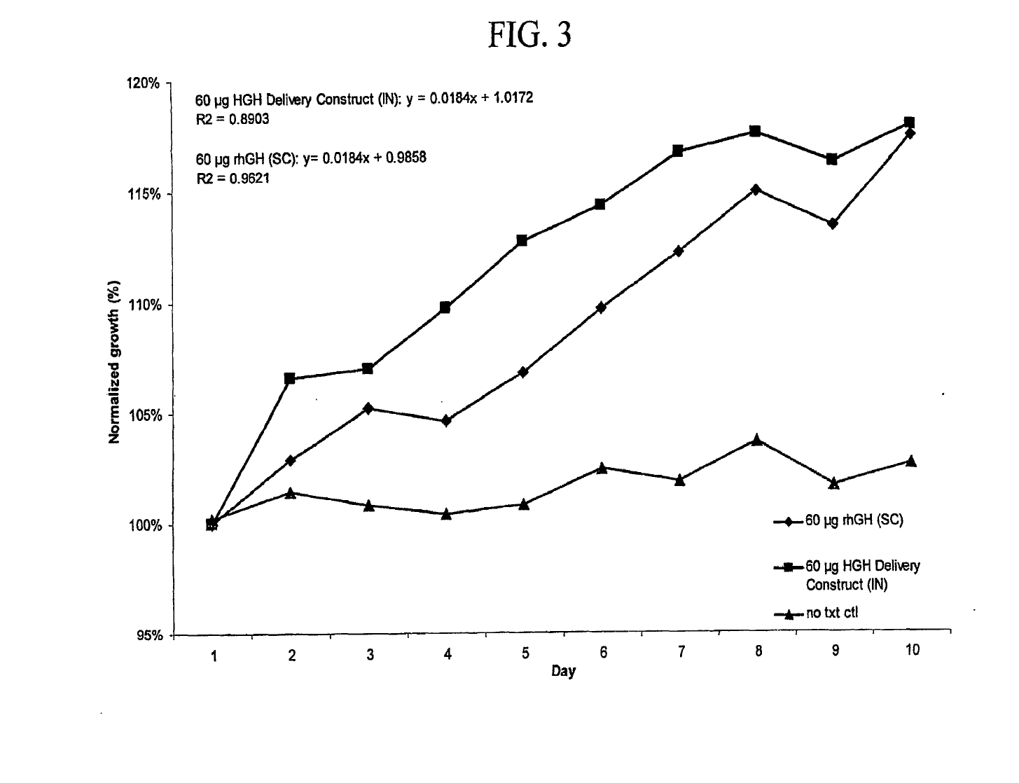

“Pseudomonas exotoxin A” or “PE” is secreted by Pseudomonas aeruginosa as a 67 kD protein composed of three prominent globular domains (Ia, II, and III) and one small subdomain (Ib) that connects domains II and III. See A. S. Allured et al., 1986, Proc. Natl. Acad. Sci. 83:1320-1324. Without intending to be bound to any particular theory or mechanism of action, domain Ia of PE is believed to mediate cell binding because domain Ia specifically binds to the low density lipoprotein receptor-related protein (“LRP”), also known as the α2-macroglobulin receptor (“α2-MR”) and CD-91. See M. Z. Kounnas et al., 1992, J. Biol. Chem. 267:12420-23. Domain Ia spans amino acids 1-252. Domain II of PE is believed to mediate transcytosis to the interior of a cell following binding of domain Ia to the α2-MR. Domain II spans amino acids 253-364. Certain portions of this domain may be required for secretion of PE from Pseudomonas aeruginosa after its synthesis. See, e.g., Vouloux et al., 2000, J. Bacterol. 182:4051-8. Domain Ib has no known function and spans amino acids 365-399. Domain III mediates cytotoxicity of PE and includes an endoplasmic reticulum retention sequence. PE cytotoxicity is believed to result from ADP ribosylation of elongation factor 2, which inactivates protein synthesis. Domain III spans amino acids 400-613 of PE. Deleting amino acid E553 (“ΔE553”) from domain III eliminates EF2 ADP ribosylation activity and detoxifies PE. PE having the mutation ΔE553 is referred to herein as “PEΔE553.” Genetically modified forms of PE are described in, e.g., U.S. Pat. Nos. 5,602,095; 5,512,658 and 5,458,878 Pseudomonas exotoxin, as used herein, also includes genetically modified, allelic, and chemically inactivated forms of PE within this definition. See, e.g., Vasil et al., 1986, Infect. Immunol. 52:538-48. Further, reference to the various domains of PE is made herein to the reference PE sequence presented as FIG. 3. However, one or more domain from modified PE, e.g., genetically or chemically modified PE, or a portion of such domains, can also be used in the chimeric immunogens of the invention so long as the domains retain functional activity. One of skill in the art can readily identify such domains of such modified PE based on, for example, homology to the PE sequence exemplified in FIG. 3 and test for functional activity using, for example, the assays described below.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide'sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60, 70, 80, 85, 90, 95, 98% or more sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60, 70, 80, 85, 90, 95, 98% or more sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gin (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), His (H) and Lys (K).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Amplification” refers to any means by which a polynucleotide sequence is copied and thus expanded into a larger number of polynucleotide molecules, e.g., by reverse transcription, polymerase chain reaction, ligase chain reaction, and the like.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

“Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, N.Y.; Sambrook et al., 2001, Molecular Cloning: A Laboratory Manual, Cold Spring Harbor Laboratory, 3^{rd} ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe.

One example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1×SSC at 45° C. for 15 minutes. An exemplary low stringency wash for a duplex of, e.g., more than about 100 nucleotides, is 4-6×SSC at 40° C. for 15 minutes. In general, a signal to noise ratio of 2× (or higher) than that observed for an unrelated probe in the particular hybridization assay indicates detection of a specific hybridization.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. Conventional notation is used herein to portray polypeptide sequences; the beginning of a polypeptide sequence is the amino-terminus, while the end of a polypeptide sequence is the carboxyl-terminus.

The term “protein” typically refers to large polypeptides, for example, polypeptides comprising more than about 50 amino acids. The term “protein” can also refer to dimers, trimers, and multimers that comprise more than one polypeptide.

“Conservative substitution” refers to the substitution in a polypeptide of an amino acid with a functionally similar amino acid. The following six groups each contain amino acids that are conservative substitutions for one another:
Alanine (A), Serine (S), and Threonine (T)
Aspartic acid (D) and Glutamnic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W).

The term “about,” as used herein, unless otherwise indicated, refers to a value that is no more than 10% above or below the value being modified by the term. For example, the term “about 5 μg/kg” means a range of from 4.5 μg/kg to 5.5 μg/kg. As another example, “about 1 hour” means a range of from 48 minutes to 72 minutes.

5.2. Methods for Increasing the Size of a Subject

In certain aspects, the invention provides methods for increasing the size of a subject by at least about 12%. These methods generally comprise administering an amount of a delivery construct comprising a growth hormone that is effective to increase the size of a subject to a mucous membrane of the subject to whom the GH is delivered. The delivery construct is typically administered in the form of a pharmaceutical composition, as described below.

Thus, in certain aspects, the invention provides a method for increasing the size of a subject by at least about 12%, comprising contacting an apical surface of a polarized epithelial cell of the subject with an amount of a delivery construct effective to increase the size of the subject by at least about 12%, wherein said delivery construct comprises a receptor binding domain, a transcytosis domain, a cleavable linker, and growth hormone (GH), wherein the transcytosis domain transcytoses the GH to and through the basal-lateral membrane of said epithelial cell, and wherein cleavage at said cleavable linker separates said GH from the remainder of said construct, thereby delivering the OH to the subject in an amount effective to increase the size of the subject by at least about 12%.

In certain embodiments, the receptor binding domain is selected from the group consisting of receptor binding domains from Pseudomonas exotoxin A, cholera toxin, diptheria toxin, shiga toxin, or shiga-like toxin; monoclonal antibodies; polygonal antibodies; single-chain antibodies; TGF α; EGF; IGF-I; IGF-41; IGF-III; IL-1; IL-2; IL-3; IL-6; MIP-1a; MIP-1b; MCAF; and IL-8. In certain embodiments, the receptor binding domain binds to a cell surface receptor selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

In certain embodiments, the transcytosis domain is selected from the group consisting of transcytosis domains from Pseudomonas exotoxin A, botulinum toxin, diptheria toxin, pertussis toxin, cholera toxin, heat-labile E. coli enterotoxin, shiga toxin, and shiga-like toxin.

In certain embodiments, the cleavable linker is cleavable by an enzyme that is selected from the group consisting of Cathepsin GI, Chymotrypsin I, Elastase I, Subtilisin AI, Subtilisin AII, Thrombin I, and Urokinase I. In certain embodiments, the cleavable linker comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7).

In certain embodiments, the epithelial cell is selected from the group consisting of nasal epithelial cells, oral epithelial cells, intestinal epithelial cells, rectal epithelial cells, vaginal epithelial cells, and pulmonary epithelial cells. In certain embodiments, the epithelial cell is a nasal epithelial cell. In certain embodiments, the epithelial cell is an intestinal epithelial cell.

In certain embodiments, the subject is a mouse or rat. In certain embodiments, the subject is a human.

In certain embodiments, the delivery construct contacts the apical membrane of the epithelial cell.

In certain embodiments, the size of the subject is increased by at least about 13%. In certain embodiments, the size of said subject is increased by at least about 14%. In certain embodiments, the size of said subject is increased by at least about 15%. In certain embodiments, the size of said subject is increased by at least about 16%. In certain embodiments, the size of said subject is increased by at least about 17%. In certain embodiments, the size of said subject is increased by at least about 18%. In certain embodiments, the size of said subject is increased by at least about 20%. In certain embodiments, the size of said subject is increased by at least about 22%. In certain embodiments, the size of said subject is increased by at least about 25%. In certain embodiments, the size of said subject is increased by at least about 27%. In certain embodiments, the size of said subject is increased by at least about 30%. In certain embodiments, the size of said subject is increased by at least about 32%. In certain embodiments, the size of said subject is increased by at least about 35%. In certain embodiments, the size of said subject is increased by at least about 37%. In certain embodiments, the size of said subject is increased by at least about 40%. In certain embodiments, the size of said subject is increased by at least about 42%. In certain embodiments, the size of said subject is increased by at least about 45%. In certain embodiments, the size of said subject is increased by at least about 47%. In certain embodiments, the size of said subject is increased by at least about 50%.

In certain embodiments, the size of said subject is increased by between about 12% and about 18%. In certain embodiments, the size of said subject is increased by between about 2% and about 50%. In certain embodiments, the size of said subject is increased by between about 2% and about 40%. In certain embodiments, the size of said subject is increased by between about 2% and about 30%. In certain embodiments, the size of said subject is increased by between about 2% and about 20%. In certain embodiments, the size of said subject is increased by between about 8% and about 50%. In certain embodiments, the size of said subject is increased by between about 8% and about 40%. In certain embodiments, the size of said subject is increased by between about 8% and about 30%. In certain embodiments, the size of said subject is increased by between about 8% and about 20%. In certain embodiments, the size of said subject is increased by between about 12% and about 50%. In certain embodiments, the size of said subject is increased by between about 12% and about 40%. In certain embodiments, the size of said subject is increased by between about 12% and about 30%. In certain embodiments, the size of said subject is increased by between about 12% and about 20%.

In certain embodiments, the size of said subject that is increased is a weight of said subject. In certain embodiments, the size of said subject that is increased is a height of said subject. In certain embodiments, the size of said subject that is increased is a length of said subject.

In certain embodiments, the GH is human growth hormone (hGH). In certain embodiments, the hGH has an amino acid sequence that is SEQ ID NO.:8.

In certain embodiments, the methods further comprise performing a method of the invention a second time about 1 day after the method of the invention is performed the first time. In certain embodiments, the methods further comprise performing a method of the invention a second time about 2 days after the method of the invention is performed the first time. In certain embodiments, the methods further comprise performing a method of the invention a second time about 3 days after the method of the invention is performed the first time.

In certain embodiments, the invention provides a method for delivering a GH to the bloodstream of a subject that results in at least about 30% bioavailability of the GH, comprising administering a delivery construct comprising the GH to the subject, thereby delivering at least about 30% of the total GH administered to the blood of the subject in a bioavailable form of the GH. In certain embodiments, at least about 10% of the total GH administered is bioavailable to the subject. In certain embodiments, at least about 15% of the total GH administered is bioavailable to the subject. In certain embodiments, at least about 20% of the total GH administered is bioavailable to the subject. In certain embodiments, at least about 25% of the total GH administered is bioavailable to the subject. In certain embodiments, at least about 35% of the total GH administered is bioavailable to the subject. In certain embodiments, at least about 40% of the total GH administered is bioavailable to the subject. In certain embodiments, at least about 45% of the total GH administered is bioavailable to the subject. In certain embodiments, at least about 50% of the total GH administered is bioavailable to the subject. In certain embodiments, at least about 55% of the total GH administered is bioavailable to the subject. In certain embodiments, at least about 60% of the total GH administered is bioavailable to the subject. In certain embodiments, at least about 65% of the total GH administered is bioavailable to the subject. In certain embodiments, at least about 70% of the total GH administered is bioavailable to the subject. In certain embodiments, at least about 75% of the total GH administered is bioavailable to the subject. In certain embodiments, at least about 80% of the total GH administered is bioavailable to the subject. In certain embodiments, at least about 85% of the total GH administered is bioavailable to the subject. In certain embodiments, at least about 90% of the total GH administered is bioavailable to the subject. In certain embodiments, at least about 95% of the total GH administered is bioavailable to the subject. In certain embodiments, the percentage of bioavailability of the GH is determined by comparing the amount of GH present in a subject's blood following administration of a delivery construct comprising the GH to the amount of GH present in a subject's blood following administration of the GH through another route of administration. In certain embodiments, the other route of administration is injection, e.g., subcutaneous injection, intravenous injection, intra-arterial injection, etc. In other embodiments, the percentage of bioavailability of the GH is determined by comparing the amount of GH present in a subject's blood following administration of a delivery construct comprising the GH to the total amount of GH administered as part of the delivery construct.

In certain embodiments, peak plasma concentrations of the delivered GH in the subject are achieved about 10 minutes after administration. In certain embodiments, peak plasma concentrations of the delivered GH in the subject are achieved about 15 minutes after administration. In certain embodiments, peak plasma concentrations of the delivered GH in the subject are achieved about 5 minutes after administration. In certain embodiments, peak plasma concentrations of the delivered GH in the subject are achieved about 20 minutes after administration. In certain embodiments, peak plasma concentrations of the delivered GH in the subject are achieved about 25 minutes after administration. In certain embodiments, peak plasma concentrations of the delivered GH in the subject are achieved about 30 minutes after administration. In certain embodiments, peak plasma concentrations of the delivered GH in the subject are achieved about 35 minutes after administration. In certain embodiments, peak plasma concentrations of the delivered GH in the subject are achieved about 40 minutes after administration. In certain embodiments, peak plasma concentrations of the delivered GH in the subject are achieved about 45 minutes after administration. In certain embodiments, peak plasma concentrations of the delivered GH in the subject are achieved about 50 minutes after administration. In certain embodiments, peak plasma concentrations of the delivered GH in the subject are achieved about 55 minutes after administration. In certain embodiments, peak plasma concentrations of the delivered GH in the subject are achieved about 60 minutes after administration. In certain embodiments, peak plasma concentrations of the delivered GH in the subject are achieved about 90 minutes after administration. In certain embodiments, peak plasma concentrations of the delivered GH in the subject are achieved about 120 minutes after administration.

In certain embodiments, the peak plasma concentration of the delivered GH is between about 0.01 ng/ml plasma and about 10 μg/ml plasma. In certain embodiments, the peak plasma concentration of the delivered GH is between about 0.01 ng/ml plasma and about 1 μg/ml plasma. In certain embodiments, the peak plasma concentration of the delivered GH is between about 0.01 ng/ml plasma and about 0.1 μg/ml plasma. In certain embodiments, the peak plasma concentration of the delivered GH is between about 0.01 ng/ml plasma and about 10 ng/ml plasma. In certain embodiments, the peak plasma concentration of the delivered GH is between about 1 ng/ml plasma and about 10 μg/ml plasma. In certain embodiments, the peak plasma concentration of the delivered GH is between about 1 ng/ml plasma and about 1 μg/ml plasma. In certain embodiments, the peak plasma concentration of the delivered GH is between about 1 ng/ml plasma and about 0.5 μg/ml plasma. In certain embodiments, the peak plasma concentration of the delivered GH is between about 1 ng/ml plasma and about 0.1 μg/ml plasma. In certain embodiments, the peak plasma concentration of the delivered GH is between about 10 ng/ml plasma and about 1 μg/ml plasma. In certain embodiments, the peak plasma concentration of the delivered GH is between about 10 ng/ml plasma and about 0.5 μg/ml plasma.

In certain embodiments, the peak plasma concentration of the delivered GH is at least about 10 μg/ml plasma. In certain embodiments, the peak plasma concentration of the delivered GH is at least about 5 μg/ml plasma. In certain embodiments, the peak plasma concentration of the delivered GH is at least about 1 μg/ml plasma. In certain embodiments, the peak plasma concentration of the delivered GH is at least about 500 ng/ml plasma. In certain embodiments, the peak plasma concentration of the delivered GH is at least about 250 ng/ml plasma. In certain embodiments, the peak plasma concentration of the delivered GH is at least about 100 ng/ml plasma. In certain embodiments, the peak plasma concentration of the delivered GH is at least about 50 ng/ml plasma. In certain embodiments, the peak plasma concentration of the delivered GH is at least about 10 ng/ml plasma. In certain embodiments, the peak plasma concentration of the delivered GH is at least about 5 ng/ml plasma. In certain embodiments, the peak plasma concentration of the delivered GH is at least about 1 ng/ml plasma. In certain embodiments, the peak plasma concentration of the delivered GH is at least about 0.1 ng/ml plasma.

In certain embodiments, the subject is a mammal. In further embodiments, the subject is a rodent, a lagomorph, or a primate. In yet further embodiments, the rodent is a mouse or rat. In other embodiments, the lagomorph is a rabbit. In still other embodiments, the primate is a human, monkey, or ape. In a preferred embodiment, the subject is a human.

5.3. Delivery Constructs

Generally, the delivery constructs of the present invention are polypeptides that have structural domains corresponding to domains Ia and II of PE. These structural domains perform certain functions, including, but not limited to, cell recognition and transcytosis, that correspond to the functions of the domains of PE.

In addition to the portions of the molecule that correspond to PE functional domains, the delivery constructs useful in the methods of this invention further comprise a growth hormone for delivery to a biological compartment of a subject. The GH can be introduced into any portion of the delivery construct that does not disrupt a cell-binding or transcytosis activity. The GH is connected with the remainder of the delivery construct with a cleavable linker.

Accordingly, the delivery constructs of the invention generally comprise the following structural elements, each element imparting particular functions to the delivery construct: (1) a “receptor binding domain” that functions as a ligand for a cell surface receptor and that mediates binding of the construct to a cell; (2) a “transcytosis domain” that mediates transcytosis from a lumen bordering the apical surface of a mucous membrane to the basal-lateral side of a mucous membrane; (3) the growth hormone; and (4) a cleavable linker that connects the GH to the remainder of the delivery construct.

The delivery constructs of the invention offer several advantages over conventional techniques for local or systemic delivery of GH to a subject. Foremost among such advantages is the ability to deliver the GH without using a needle to puncture the skin of the subject. Many subjects require repeated, regular doses of GH. Such subjects' quality of life would be greatly improved if the delivery of GH could be accomplished without injection, by avoiding pain or potential complications associated therewith.

In addition, connection of the GH to the remainder of the delivery construct with a linker that is cleaved by an enzyme present at a basal-lateral membrane of an epithelial cell allows the GH to be liberated from the delivery construct and released from the remainder of the delivery construct soon after transcytosis across the epithelial membrane. Such liberation reduces the probability of induction of an immune response against the GH. It also allows the GH to interact with its target free from the remainder of the delivery construct.

Other advantages of the delivery constructs of the invention will be apparent to those of skill in the art.

In certain embodiments, the invention provides a delivery construct that comprises a receptor binding domain, a transcytosis domain, a growth hormone to be delivered to a subject, and a cleavable linker. Cleavage at the cleavable linker separates the GH from the remainder of the construct. The cleavable linker can be cleavable by an enzyme that is present at a basal-lateral membrane of a polarized epithelial cell of the subject or in the plasma of the subject. In certain embodiments, the enzyme that is at a basal-lateral membrane of a polarized epithelial cell exhibits higher activity on the basal-lateral side of a polarized epithelial cell than it does on the apical side of the polarized epithelial cell. In certain embodiments, the enzyme that is in the plasma of the subject exhibits higher activity in the plasma than it does on the apical side of a polarized epithelial cell.

In certain embodiments, the delivery construct further comprises a second cleavable linker. In certain embodiments, the first and/or the second cleavable linker comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7). In certain embodiments, the first and/or the second cleavable linker comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7) and is cleavable by an enzyme that exhibits higher activity on the basal-lateral side of a polarized epithelial cell than it does on the apical side of the polarized epithelial cell. In certain embodiments, the first and/or the second cleavable linker comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7) and is cleavable by an enzyme that exhibits higher activity in the plasma than it does on the apical side of a polarized epithelial cell.

In certain embodiments, the enzyme that is present at a basal-lateral membrane of a polarized epithelial cell is selected from the group consisting of Cathepsin GI, Chymotrypsin I, Elastase I, Subtilisin AI, Subtilisin AII, Thrombin I, and Urokinase I.

In certain embodiments, the receptor binding domain is selected from the group consisting of receptor binding domains from Pseudomonas exotoxin A, cholera toxin, botulinum toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain binds to a cell-surface receptor that is selected from the group consisting of α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor. In further embodiments, the receptor binding domain of Pseudomonas exotoxin A is Domain Ia of Pseudomonas exotoxin A. In yet further embodiments, the receptor binding domain of Pseudomonas exotoxin A has an amino acid sequence that is SEQ ID NO.:9.

In certain embodiments, the transcytosis domain is selected from the group consisting of transcytosis domains from Pseudomonas exotoxin A, botulinum toxin, diptheria toxin, pertussis toxin, cholera toxin, heat-labile E. coli enterotoxin, shiga toxin, and shiga-like toxin. In further embodiments, the transcytosis domain is Pseudomonas exotoxin A transcytosis domain. In still further embodiments, the Pseudomonas exotoxin A transcytosis domain has an amino acid sequence that is SEQ ID NO.:10.

5.3.1. Receptor Binding Domain

The delivery constructs of the invention generally comprise a receptor binding domain. The receptor binding domain can be any receptor binding domain known to one of skill in the art without limitation to bind to a cell surface receptor that is present on the apical membrane of an epithelial cell. Preferably, the receptor binding domain binds specifically to the cell surface receptor. The receptor binding domain should bind to the cell surface receptor with sufficient affinity to allow endocytosis of the delivery construct.

In certain embodiments, the receptor binding domain can comprise a peptide, a polypeptide, a protein, a lipid, a carbohydrate, or a small organic molecule, or a combination thereof. Examples of each of these molecules that bind to cell surface receptors present on the apical membrane of epithelial cells are well known to those of skill in the art. Suitable peptides or polypeptides include, but are not limited to, bacterial toxin receptor binding domains, such as the receptor binding domains from PE, cholera toxin, botulinum toxin, diptheria toxin, shiga toxin, shiga-like toxin, etc.; antibodies, including monoclonal, polyclonal, and single-chain antibodies, or derivatives thereof, growth factors, such as EGF, IGF-I, IGF-II, IGF-III etc.; cytokines, such as IL-1, IL-2, IL-3, IL-6, etc; chemokines, such as MIP-1a, MIP-1b, MCAF, IL-8, etc.; and other ligands, such as CD4, cell adhesion molecules from the immunoglobulin superfamily, integrins, ligands specific for the IgA receptor, etc. See, e.g., Pastan et al., 1992, Annu. Rev. Biochem. 61:331-54; and U.S. Pat. Nos. 5,668,255, 5,696,237, 5,863,745, 5,965,406, 6,022,950, 6,051,405, 6,251,392, 6,440,419, and 6,488,926. The skilled artisan can select the appropriate receptor binding domain based upon the expression pattern of the receptor to which the receptor binding domain binds.

Lipids suitable for receptor binding domains include, but are not limited to, lipids that themselves bind cell surface receptors, such as sphingosine-1-phosphate, lysophosphatidic acid, sphingosylphosphorylcholine, retinoic acid, etc.; lipoproteins such as apolipoprotein E, apolipoprotein A, etc., and glycolipids such as lipopolysaccharide, etc.; glycosphingolipids such as globotriaosylceramide and galabiosylceramide; and the like. Carbohydrates suitable for receptor binding domains include, but are not limited to, monosaccharides, disaccharides, and polysaccharides that comprise simple sugars such as glucose, fructose, galactose, etc.; and glycoproteins such as mucins, selecting, and the like. Suitable small organic molecules for receptor binding domains include, but are not limited to, vitamins, such as vitamin A, B₁, B₂, B₃, B₆, B₉, B₁₂, C, D, E, and K, amino acids, and other small molecules that are recognized and/or taken up by receptors present on the apical surface of epithelial cells. U.S. Pat. No. 5,807,832 provides an example of such small organic molecule receptor binding domains, vitamin B₁₂.

In certain embodiments, the receptor binding domain can bind to a receptor found on an epithelial cell. In further embodiments, the receptor binding domain can bind to a receptor found on the apical membrane of an epithelial cell. The receptor binding domain can bind to any receptor known to be present on the apical membrane of an epithelial cell by one of skill in the art without limitation. For example, the receptor binding domain can bind to α2-MR, EGFR, or IGFR. An example of a receptor binding domain that can bind to α2-MR is domain Ia of PE. Accordingly, in certain embodiments, the receptor binding domain is domain Ia of PE. In other embodiments, the receptor binding domain is a portion of domain Ia of PE that can bind to α2-MR. Exemplary receptor binding domains that can bind to EGFR include, but are riot limited to, EGF and TGFα. Examples of receptor binding domains that can bind to IGFR include, but are not limited to, IGF-I, IGF-II, or IGF-III. Thus, in certain embodiments, the receptor binding domain is EGF, IGF-I, IGF-II, or IGF-III. In other embodiments, the receptor binding domain is a portion of EGF, IGF-I, IGF-II, or IGF-III that can bind to the EGF or IGF receptor.

In certain embodiments, the receptor binding domain binds to a receptor that is highly expressed on the apical membrane of a polarized epithelial cell but is not expressed or expressed at low levels on antigen presenting cells, such as, for example, dendritic cells. Exemplary receptor binding domains that have this kind of expression pattern include, but are not limited to, TGFα, EGF, IGF-I, IGF-II, and IGF-III.

In certain embodiments, the delivery constructs of the invention comprise more than one domain that can function as a receptor binding domain. For example, the delivery construct can comprise PE domain Ia in addition to another receptor binding domain.

The receptor binding domain can be attached to the remainder of the delivery construct by any method or means known by one of skill in the art to be useful for attaching such molecules, without limitation. In certain embodiments, the receptor binding domain is expressed together with the remainder of the delivery construct as a fusion protein. Such embodiments are particularly usefull when the receptor binding domain and the remainder of the construct are formed from peptides or polypeptides.

In other embodiments, the receptor binding domain is connected with the remainder of the delivery construct with a linker. In yet other embodiments, the receptor binding domain is connected with the remainder of the delivery construct without a linker. Either of these embodiments are useful when the receptor binding domain comprises a peptide, polypeptide, protein, lipid, carbohydrate, nucleic acid, or small organic molecule.

In certain embodiments, the linker can form a covalent bond between the receptor binding domain and the remainder of the delivery construct. In certain embodiments, the covalent bond can be a peptide bond. In other embodiments, the linker can link the receptor binding domain to the remainder of the delivery construct with one or more non-covalent interactions of sufficient affinity. One of skill in the art can readily recognize linkers that interact with each other with sufficient affinity to be useful in the delivery constructs of the invention. For example, biotin can be attached to the receptor binding domain, and streptavidin can be attached to the remainder of the molecule. In certain embodiments, the linker can directly link the receptor binding domain to the remainder of the molecule. In other embodiments, the linker itself comprises two or more molecules that associate in order to link the receptor binding domain to the remainder of the molecule. Exemplary linkers include, but are not limited to, straight or branched-chain carbon linkers, heterocyclic carbon linkers, substituted carbon linkers, unsaturated carbon linkers, aromatic carbon linkers, peptide linkers, etc.

In embodiments where a linker is used to connect the receptor binding domain to the remainder of the delivery construct, the linkers can be attached to the receptor binding domain and/or the remainder of the delivery construct by any means or method known by one of skill in the art without limitation. For example, the linker can be attached to the receptor binding domain and/or the remainder of the delivery construct with an ether, ester, thioether, thioester, amide, imide, disulfide, peptide, or other suitable moiety. The skilled artisan can select the appropriate linker and method for attaching the linker based on the physical and chemical properties of the chosen receptor binding domain and the linker. The linker can be attached to any suitable functional group on the receptor binding domain or the remainder of the molecule. For example, the linker can be attached to sulfhydryl (—S), carboxylic acid (COOH) or free amine (—NH2) groups, which are available for reaction with a suitable functional group on a linker. These groups can also be used to connect the receptor binding domain directly connected with the remainder of the molecule in the absence of a linker.

Further, the receptor binding domain and/or the remainder of the delivery construct can be derivatized in order to facilitate attachment of a linker to these moieties. For example, such derivatization can be accomplished by attaching suitable derivative such as those available from Pierce Chemical Company, Rockford, Ill. Alternatively, derivatization may involve chemical treatment of the receptor binding domain and/or the remainder of the molecule. For example, glycol cleavage of the sugar moiety of a carbohydrate or glycoprotein receptor binding domain with periodate generates free aldehyde groups. These free aldehyde groups may be reacted with free amine or hydrazine groups on the remainder of the molecule in order to connect these portions of the molecule. See, e.g., U.S. Pat. No. 4,671,958. Further, the skilled artisan can generate free sulfhydryl groups on proteins to provide a reactive moiety for making a disulfide, thioether, thioester, etc. linkage. See, e.g., U.S. Pat. No. 4,659,839.

Any of these methods for attaching a linker to a receptor binding domain and/or the remainder of a delivery construct can also be used to connect a receptor binding domain with the remainder of the delivery construct in the absence of a linker. In such embodiments, the receptor binding domain is coupled with the remainder of the construct using a method suitable for the particular receptor binding domain. Thus, any method suitable for connecting a protein, peptide, polypeptide, nucleic acid, carbohydrate, lipid, or small organic molecule to the remainder of the delivery construct known to one of skill in the art, without limitation, can be used to connect the receptor binding domain to the remainder of the construct. In addition to the methods for attaching a linker to a receptor binding domain or the remainder of a delivery construct, as described above, the receptor binding domain can be connected with the remainder of the construct as described, for example, in U.S. Pat. Nos. 6,673,905; 6,585,973; 6,596,475; 5,856,090; 5,663,312; 5,391,723; 6,171,614; 5,366,958; and 5,614,503.

In certain embodiments, the receptor binding domain can be a monoclonal antibody. In some of these embodiments, the chimeric immunogen is expressed as a fusion protein that comprises an immunoglobulin heavy chain from an immunoglobulin specific for a receptor on a cell to which the chimeric immunogen is intended to bind. The light chain of the immunoglobulin then can be co-expressed with the chimeric immunogen, thereby forming a light chain-heavy chain dimer. In other embodiments, the antibody can be expressed and assembled separately from the remainder of the chimeric immunogen and chemically linked thereto.

5.3.2. Transcytosis Domain

The delivery constructs of the invention also comprise a transcytosis domain. The transcytosis domain can be any transcytosis domain known by one of skill in the art to effect transcytosis of chimeric proteins that have bound to a cell surface receptor present on the apical membrane of an epithelial cell. In certain embodiments, the transcytosis domain is a transcytosis domain from PE, diptheria toxin, pertussis toxin, cholera toxin, heat-labile E. coli enterotoxin, shiga toxin, or shiga-like toxin. See, for example, U.S. Pat. Nos. 5,965,406, and 6,022,950. In preferred embodiments, the transcytosis domain is domain II of PE.

The transcytosis domain need not, though it may, comprise the entire amino acid sequence of domain II of native PE, which spans residues 253-364 of PE. For example, the transcytosis domain can comprise a portion of PE that spans residues 280-344 of domain II of PE. The amino acids at positions 339 and 343 appear to be necessary for transcytosis. See Siegall et al., 1991, Biochemistry 30:7154-59. Further, conservative or nonconservative substitutions can be made to the amino acid sequence of the transcytosis domain, as long as transcytosis activity is not substantially eliminated. A representative assay that can routinely be used by one of skill in the art to determine whether a transcytosis domain has transcytosis activity is described below.

Without intending to be limited to any particular theory or mechanism of action, the transcytosis domain is believed to permit the trafficking of the delivery construct through a polarized epithelial cell after the construct binds to a receptor present on the apical surface of the polarized epithelial cell. Such trafficking through a polarized epithelial cell is referred to herein as “transcytosis.” This trafficking permits the release of the delivery construct from the basal-lateral membrane of the polarized epithelial cell.

5.3.3. Growth Hormones for Delivery

The delivery constructs of the invention also comprise a growth hormone. The GH can be attached to the remainder of the delivery construct by any method known by one of skill in the art, without limitation. In certain embodiments, the GH is expressed together with the remainder of the delivery construct as a fusion protein. In such embodiments, the GH can be inserted into or attached to any portion of the delivery construct, so long as the receptor binding domain, the transcytosis domain, and GH retain their activities. The GH is connected with the remainder of the construct with a cleavable linker, or a combination of cleavable linkers, as described below.

In native PE, the Ib loop (domain Ib) spans amino acids 365 to 399, and is structurally characterized by a disulfide bond between two cysteines at positions 372 and 379. This portion of PE is not essential for any known activity of PE, including cell binding, transcytosis, ER retention or ADP ribosylation activity. Accordingly, domain Ib can be deleted entirely, or modified to contain GH.

Thus, in certain embodiments, the GH can be inserted into domain Ib. If desirable, the GH can be inserted into domain Ib wherein the cysteines at positions 372 and 379 are not cross-linked. This can be accomplished by reducing the disulfide linkage between the cysteines, by deleting the cysteines entirely from the Ib domain, by mutating the cysteines to other residues, such as, for example, serine, or by other similar techniques. Alternatively, the OH can be inserted into the Ib loop between the cysteines at positions 372 and 379. In such embodiments, the disulfide linkage between the cysteines can be used to constrain the GH if desirable. In any event, in embodiments where the GH is inserted into domain Ib of PE, or into any other portion of the delivery construct, the GH should be flanked by cleavable linkers such that cleavage at the cleavable linkers liberates the GH from the remainder of the construct.

In other embodiments, the GH can be connected with the N-terminal or C-terminal end of a polypeptide portion of the delivery construct. In such embodiments, the method of connection should be designed to avoid interference with other functions of the delivery construct, such as receptor binding or transcytosis. In yet other embodiments, the GH can be connected with a side chain of an amino acid of the delivery construct. The GH is connected with the remainder of the delivery construct with a cleavable linker, as described below. In such embodiments, the GH to be delivered can be connected with the remainder of the delivery construct with one or more cleavable linkers such that cleavage at the cleavable linker(s) separates the GH from the remainder of the delivery construct. It should be noted that, in certain embodiments, the GH of interest can also comprise a short (1-20 amino acids, preferably 1-10 amino acids, and more preferably 1-5 amino acids) leader peptide in addition to the GH of interest that remains attached to the GH following cleavage of the cleavable linker. Preferably, this leader peptide does not affect the activity or immunogenicity of the GH. Even more preferably, the cleavable linker is selected such that cleavage of the cleavable linker releases the GH in its mature, native, active form without any amino acids present in the released GH that are not present in endogenously produced mature GH.

In embodiments where the GH is expressed together with another portion of the delivery construct as a fusion protein, the GH can be can be inserted into the delivery construct by any method known to one of skill in the art without limitation. For example, amino acids corresponding to the GH can be inserted directly into the delivery construct, with or without deletion of native amino acid sequences. In certain embodiments, all or part of the Ib domain of PE can be deleted and replaced with the GH. In certain embodiments, the cysteine residues of the Ib loop are deleted so that the GH remains unconstrained. In other embodiments, the cysteine residues of the Ib loop are linked with a disulfide bond and constrain the GH.

The GH can be any GH that is desired to be introduced into a subject. Thus, the GH can be a human GH, a mouse GH, a rat GH, and the like. Preferably, the GH is matched to the subject to whom the GH is to be administered, for example, if the GH is to be administered to a human, the GH is preferably human GH.

In certain embodiments, the GH can be selected to not be cleavable by an enzyme present at the basal-lateral membrane of an epithelial cell. For example, the assays described in the examples can be used to routinely test whether such a cleaving enzyme can cleave the GH to be delivered. If so, the GH can be routinely altered to eliminate the offending amino acid sequence recognized by the cleaving enzyme. The altered GH can then be tested to ensure that it retains activity using methods routine in the art.

5.3.4. Cleavable Linkers

In the delivery constructs of the invention, the GH to be delivered to the subject is connected with the remainder of the delivery construct with one or more cleavable linkers. The number of cleavable linkers present in the construct depends, at least in part, on the location of the OH in relation to the remainder of the delivery construct and the nature of the GH. When the GH is inserted into the delivery construct, the GH can be flanked by cleavable linkers, such that cleavage at both linkers separates the GH. The flanking cleavable linkers can be the same or different from each other. When the GH can be separated from the remainder of the delivery construct with cleavage at a single linker, the delivery constructs can comprise a single cleavable linker.

The cleavable linkers are generally cleavable by a cleaving enzyme that is present at or near the basal-lateral membrane of an epithelial cell. By selecting the cleavable linker to be cleaved by such enzymes, the GH can be liberated from the remainder of the construct following transcytosis across the mucous membrane and release from the epithelial cell into the cellular matrix on the basal-lateral side of the membrane. Further, cleaving enzymes could be used that are present inside the epithelial cell, such that the cleavable linker is cleaved prior to release of the delivery construct from the basal-lateral membrane, so long as the cleaving enzyme does not cleave the delivery construct before the delivery construct enters the trafficking pathway in the polarized epithelial cell that results in release of the delivery construct and GH from the basal-lateral membrane of the cell.

In certain embodiments, the cleaving enzyme is a peptidase. In other embodiments, the cleaving enzyme is an RNAse. In yet other embodiments, the cleaving enzyme can cleave carbohydrates. Preferred peptidases include, but are not limited to, Cathepsin GI, Chymotrypsin I, Elastase I, Subtilisin AI, Subtilisin AII, Thrombin I, and Urokinase I. Table 1 presents these enzymes together with an amino acid sequence that is recognized and cleaved by the particular peptidase.

TABLE 1

Peptidases Present Near Basal-Lateral Mucous
Membranes

| | |
|---|---|
| | Amino Acid Sequence Recognized and |
| Peptidase | Cleaved |

| | | |
|---|---|---|
| Cathepsin GI | Ala-Ala-Pro-Phe | (SEQ ID NO.: 1) |
| Chymotrypsin I | Gly-Gly-Phe | (SEQ ID NO.: 2) |
| Elastase I | Ala-Ala-Pro-Val | (SEQ ID NO.: 3) |
| Subtilisin AI | Gly-Gly-Leu | (SEQ ID NO.: 4) |
| Subtilisin AII | Ala-Ala-Leu | (SEQ ID NO.: 5) |
| Thrombin I | Phe-Val-Arg | (SEQ ID NO.: 6) |
| Urokinase I | Val-Gly-Arg | (SEQ ID NO.: 7) |

In certain embodiments, the delivery construct can comprise more than one cleavable linker, wherein cleavage at either cleavable linker can separate the GH to be delivered from the delivery construct. In certain embodiments, the cleavable linker can be selected to avoid the use of cleavable linkers that comprise sequences present in the GH to be delivered. For example, if the GH comprises AAL, the cleavable linker can be selected to be cleaved by an enzyme that does not recognize this sequence.

Further, the cleavable linker preferably exhibits a greater propensity for cleavage than the remainder of the delivery construct. As one skilled in the art is aware, many peptide and polypeptide sequences can be cleaved by peptidases and proteases. In certain embodiments, the cleavable linker is selected to be preferentially cleaved relative to other amino acid sequences present in the delivery construct during administration of the delivery construct. In certain embodiments, the receptor binding domain is substantially (e.g., about 99%, about 95%, about 90%, about 85%, about 80, or about 75%) intact following delivery of the delivery construct to the bloodstream of the subject. In certain embodiments, the translocation domain is substantially (e.g., about 99%, about 95%, about 90%, about 85%, about 80, or about 75%) intact following delivery of the delivery construct to the bloodstream of the subject. In certain embodiments, the GH is substantially (e.g., about 99%, about 95%, about 90%, about 85%, about 80, or about 75%) intact following delivery of the delivery construct to the bloodstream of the subject. In certain embodiments, the cleavable linker is substantially (e.g., about 99%, about 95%, about 90%, about 85%, about 80, or about 75%) cleaved following delivery of the delivery construct to the bloodstream of the subject.

In other embodiments, the cleavable linker is cleaved by a cleaving enzyme found in the plasma of the subject. Any cleaving enzyme known by one of skill in the art to be present in the plasma of the subject can be used to cleave the cleavable linker. Use of such enzymes to cleave the cleavable linkers is less preferred than use of cleaving enzymes found near the basal-lateral membrane of a polarized epithelial cell because it is believed that more efficient cleavage will occur in near the basal-lateral membrane. However, if the skilled artisan determines that cleavage mediated by a plasma enzyme is sufficiently efficient to allow cleavage of a sufficient fraction of the delivery constructs to avoid adverse effects, such plasma cleaving enzymes can be used to cleave the delivery constructs. Accordingly, in certain embodiments, the cleavable linker can be cleaved with an enzyme that is selected from the group consisting of caspase-1, caspase-3, proprotein convertase 1, proprotein convertase 2, proprotein convertase 4, proprotein convertase 4 PACE 4, prolyl oligopeptidase, endothelin cleaving enzyme, dipeptidyl-peptidase IV, signal peptidase, neprilysin, renin, and esterase. See, e.g., U.S. Pat. No. 6,673,574. Table 2 presents these enzymes together with an amino acid sequence(s) recognized by the particular peptidase. The peptidase cleaves a peptide comprising these sequences at the N-terminal side of the amino acid identified with an asterisk.

TABLE 2

Plasma Peptidases

| | Amino Acid Sequence |
|---|---|
| Peptidase | Recognized and Cleaved |
| Caspase-1 | Tyr-Val-Ala-Asp-Xaa* |
| | (SEQ ID NO.: 11) |
| Caspase-3 | Asp-Xaa-Xaa-Asp-Xaa* |
| | (SEQ ID NO.: 12) |
| Proprotein | Arg-(Xaa)n-Arg-Xaa*; |
| convertase 1 | n = 0, 2, 4 or 6 |
| | (SEQ ID NO.: 13) |
| Proprotein | Lys-(Xaa)n-Arg-Xaa*; |
| convertase 2 | n = 0, 2, 4, or 6 |
| | (SEQ ID NO.: 14) |
| Proprotein | Glp-Arg-Thr-Lys-Arg-Xaa* |
| convertase 4 | (SEQ ID NO.: 15) |
| Proprotein | Arg-Val-Arg-Arg-Xaa* |
| convertase 4 PACE 4 | (SEQ ID NO.: 16) |
| | Decanoyl-Arg-Val-Arg-Arg-Xaa* |
| | (SEQ ID NO.: 17) |
| Prolyloligopeptidase | Pro-Xaa*-Trp-Val-Pro-Xaa |
| Endothelin cleaving | (SEQ ID NO.: 18) |
| enzyme in combination | |
| with dipeptidyl- | |
| peptidase IV | |
| Signal peptidase | Trp-Val*-Ala-Xaa |
| | (SEQ ID NO.: 19) |
| Neprilysin in combi- | Xaa-Phe*-Xaa-Xaa |
| nation with dipep- | (SEQ ID NO.: 20) |
| tidyl-peptidase IV | Xaa-Tyr*-Xaa-Xaa |
| | (SEQ ID NO.: 21) |
| | Xaa-Trp*-Xaa-Xaa |
| | (SEQ ID NO.: 22) |
| Renin in combination | Asp-Arg-Tyr-Ile-Pro-Phe-His- |
| with dipeptidyl- | Leu*-Leu-(Val, Ala or Pro)- |
| peptidase IV | Tyr-(Ser, Pro, or Ala) |
| | (SEQ ID NO. :23) |

Thus, in certain more preferred embodiments, the cleavable linker can be any cleavable linker known by one of skill in the art to be cleavable by an enzyme that is present at the basal-lateral membrane of an epithelial cell. In certain embodiments, the cleavable linker comprises a peptide. In other embodiments, the cleavable linker comprises a nucleic acid, such as RNA or DNA. In still other embodiments, the cleavable linker comprises a carbohydrate, such as a disaccharide or a trisaccharide. In certain embodiments, the cleavable linker is a peptide that comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7).

Alternatively, in less preferred embodiments, the cleavable linker can be any cleavable linker known by one of skill in the art to be cleavable by an enzyme that is present in the plasma of the subject to whom the delivery construct is administered. In certain embodiments, the cleavable linker comprises a peptide. In other embodiments, the cleavable linker comprises a nucleic acid, such as RNA or DNA. In still other embodiments, the cleavable linker comprises a carbohydrate, such as a disaccharide or a trisaccharide. In certain embodiments, the cleavable linker is a peptide that comprises an amino acid sequence that is selected from the group consisting of amino acid sequences presented in Table 2.

In certain embodiments, the delivery construct comprises more than one cleavable linker. In certain embodiments, cleavage at any of the cleavable linkers will separate the GH to be delivered from the remainder of the delivery construct. In certain embodiments, the delivery construct comprises a cleavable linker cleavable by an enzyme present at the basal-lateral side of a polarized epithelial membrane and a cleavable linkers cleavable by an enzyme that is present in the plasma of the subject to whom the delivery construct is administered.

In other embodiments, the cleavable linker can be a cleavable linker that is cleaved following a change in the environment of the delivery construct. For example, the cleavable linker can be a cleavable linker that is pH sensitive and is cleaved by a change in pH that is experienced when the delivery construct is released from the basal-lateral membrane of a polarized epithelial cell. For instance, the intestinal lumen is strongly alkaline, while plasma is essentially neutral. Thus, a cleavable linker can be a moiety that is cleaved upon a shift from alkaline to neutral pH. The change in the environment of the delivery construct that cleaves the cleavable linker can be any environmental change that that is experienced when the delivery construct is released from the basal-lateral membrane of a polarized epithelial cell known by one of skill in the art, without limitation.

5.4. Methods of Administration

The delivery constructs of the invention can be administered to a subject by any method known to one of skill in the art. In certain embodiments, the delivery constructs are contacted to a mucosal membrane of the subject. For example, the mucosal membrane can be present in the eye, nose, mouth, trachea, lungs, esophagus, stomach, small intestine, large intestine, rectum, anus, sweat glands, vulva, vagina, or penis of the subject. Preferably, the mucosal membrane is a mucosal membrane present in the digestive tract of the subject, such as a mucosal membrane in the mouth, esophagus, stomach, small intestine, large intestine, or rectum of the subject. Nasal mucosal membranes are equally preferred.

In embodiments where the mucosal membrane is in the digestive tract of the subject, the delivery constructs are preferably administered to the subject orally. Thus, the delivery construct can be formulated to protect the delivery construct from degradation in the acid environment of the stomach, if necessary. For example, many embodiments of the delivery constructs of the invention comprise polypeptide domains with defined activities. Unless such delivery constructs are protected from acid and/or enzymatic hydrolysis in the stomach, the constructs will generally be digested before delivery of substantial amounts of the GH to be delivered. Accordingly, composition formulations that protect the delivery construct from degradation can be used in administration of these delivery constructs.

5.4.1. Dosage

Generally, an amount of the delivery construct comprising a GH effective to increase the size of a subject by at least about 12% is administered to a subject. The skilled artisan can readily determine if the dosage of the delivery construct is sufficient to deliver an effective amount of the GH, as described below. In certain embodiments, between about 1 μg and about 1 g of delivery construct is administered. In other embodiments, between about 10 μg and about 500 mg of delivery construct is administered. In still other embodiments, between about 10 μg and about 100 mg of delivery construct is administered. In yet other embodiments, between about 10 μg and about 1000 μg of delivery construct is administered. In still other embodiments, between about 10 μg and about 250 μg of delivery construct is administered. In yet other embodiments, between about 10 μg and about 100 μg of delivery construct is administered. Preferably, between about 10 μg and about 50 μg of delivery construct is administered.

The volume of a composition comprising the delivery construct that is administered will generally depend on the concentration of delivery construct and the formulation of the composition. In certain embodiments, a unit dose of the delivery construct composition is between about 0.05 ml and about 1 ml, preferably about 0.5 ml. The delivery construct compositions can be prepared in dosage forms containing between 1 and 50 doses (e.g., 0.5 ml to 25 ml), more usually between 1 and 10 doses (e.g., 0.5 ml to 5 ml)

The delivery construct compositions of the invention can be administered in one dose or in multiple doses. A dose can be followed by one or more doses spaced by about 1 to about 6 hours, by about 6 to about 12 hours, by about 12 to about 24 hours, by about 1 day to about 3 days, by about 1 day to about 1 week, by about 1 week to about 2 weeks, by about 2 weeks to about 1 month, by about 4 to about 8 weeks, by about 1 to about 3 months, or by about 1 to about 6 months.

The growth hormones to be delivered are generally growth hormones for which a large amount of knowledge regarding dosage, frequency of administration, and methods for assessing effective concentrations in subjects has accumulated. Such knowledge can be used to assess efficiency of delivery, effective concentration of the growth hormone in the subject, and frequency of administration. Thus, the knowledge of those skilled in the art can be used to determine whether, for example, the amount of GH delivered to the subject is an amount effective to increase the size of the subject by at least about 12%, the dosage should be increased or decreased to achieve this result, the subject should be administered the delivery construct more or less frequently to achieve this result, and the like.

5.4.2. Determining Amounts of Growth Hormone Delivered

The methods of the invention can be used to deliver, either locally or systemically, a pharmaceutically effective amount of a GH to a subject. The skilled artisan can determine whether the methods result in delivery of such a pharmaceutically effective amount of the GH. The exact methods will depend on the GH that is delivered, but generally will rely on either determining the concentration of the GH in the blood of the subject or in the biological compartment of the subject where the GH exerts its effects. Alternatively or additionally, the effects of the GH on the subject can be monitored.

For example, the skilled artisan can determine whether a pharmaceutically effective amount of GH had been delivered to the subject by, for example, taking a plasma sample from the subject and determining the concentration of GH therein. One exemplary method for determining the concentration of GH is by performing an ELISA assay, but any other suitable assay known to the skilled artisan can be used.

Alternatively, one of skill in the art can determine if an effective amount of GH had been delivered to the subject by monitoring any effect of a GH known by one of skill in the art, without limitation, can be assessed in determining whether an effective amount of the GH has been administered. Exemplary effects include, but are not limited to, receptor binding, receptor activation, downstream effects of receptor binding, downstream effects of receptor activation, coordination of compounds, effective blood clotting, bone growth, wound healing, cellular proliferation, weight increase, etc.

5.5. Polynucleotides Encoding Delivery Constructs

In another aspect, the invention provides polynucleotides comprising a nucleotide sequence encoding the delivery constructs. These polynucleotides are useful, for example, for making the delivery constructs. In yet another aspect, the invention provides an expression system that comprises a recombinant polynucleotide sequence encoding a receptor binding domain, a transcytosis domain, and a polylinker insertion site for a polynucleotide sequence encoding a GH. The polylinker insertion site can be anywhere in the polynucleotide sequence so long as the polylinker insertion does not disrupt the receptor binding domain or the transcytosis domain. The polylinker insertion site should be oriented near a polynucleotide sequence that encodes a cleavable linker so that cleavage at the cleavable linker separates a GH encoded by a nucleic acid inserted into the polylinker insertion site from the remainder of the encoded delivery construct. Thus, in embodiments where the polylinker insertion site is at an end of the encoded construct, the polynucleotide comprises one nucleotide sequence encoding a cleavable linker between the polylinker insertion site and the remainder of the polynucleotide. In embodiments where the polylinker insertion site is not at the end of the encoded construct, the polylinker insertion site can be flanked by nucleotide sequences that each encode a cleavable linker.

In certain embodiments, the recombinant polynucleotides are based on polynucleotides encoding PE, or portions or derivatives thereof. In other embodiments, the recombinant polynucleotides are based on polynucleotides that hybridize to a polynucleotide that encodes PE under stringent hybridization conditions. A nucleotide sequence encoding PE is presented as SEQ ID NO.:24. This sequence can be used to prepare PCR primers for isolating a nucleic acid that encodes any portion of this sequence that is desired. For example, PCR can be used to isolate a nucleic acid that encodes one or more of the functional domains of PE. A nucleic acid so isolated can then be joined to nucleic acids encoding other functional domains of the delivery constructs using standard recombinant techniques.

Other in vitro methods that can be used to prepare a polynucleotide encoding PE, PE domains, or any other functional domain useful in the delivery constructs of the invention include, but are not limited to, reverse transcription, the polymerase chain reaction (PCR), the ligase chain reaction (LCR), the transcription-based amplification system (TAS), the self-sustained sequence replication system (3SR) and the QP replicase amplification system (QB). Any such technique known by one of skill in the art to be useful in construction of recombinant nucleic acids can be used. For example, a polynucleotide encoding the protein or a portion thereof can be isolated by polymerase chain reaction of cDNA using primers based on the DNA sequence of PE or a nucleotide encoding a receptor binding domain.

Guidance for using these cloning and in vitro amplification methodologies are described in, for example, U.S. Pat. No. 4,683,195; Mullis et al., 1987, Cold Spring Harbor Symp. Quant. Biol.51:263; and Erlich, ed., 1989, PCR Technology, Stockton Press, NY. Polynucleotides encoding a delivery construct or a portion thereof also can be isolated by screening genomic or cDNA libraries with probes selected from the sequences of the desired polynucleotide under stringent, moderately stringent, or highly stringent hybridization conditions.

Construction of nucleic acids encoding the delivery constructs of the invention can be facilitated by introducing an insertion site for a nucleic acid encoding the GH into the construct. In certain embodiments, an insertion site for the GH can be introduced between the nucleotides encoding the cysteine residues of domain Ib. In other embodiments, the insertion site can be introduced anywhere in the nucleic acid encoding the construct so long as the insertion does not disrupt the functional domains encoded thereby. In certain embodiments, the insertion site can be in the ER retention domain.

In more specific embodiments, a nucleotide sequence encoding a portion of the Ib domain between the cysteine-encoding residues can be removed and replaced with a nucleotide sequence that includes a cloning site cleaved by a restriction enzyme. For example, the cloning site can be recognized and cleaved by PstI. In such examples, a polynucleotide encoding GH that is flanked by PstI sequences can be inserted into the vector.

Further, the polynucleotides can also encode a secretory sequence at the amino terminus of the encoded delivery construct. Such constructs are useful for producing the delivery constructs in mammalian cells as they simplify isolation of the immunogen.

Furthermore, the polynucleotides of the invention also encompass derivative versions of polynucleotides encoding a delivery construct. Such derivatives can be made by any method known by one of skill in the art without limitation. For example, derivatives can be made by site-specific mutagenesis, including substitution, insertion, or deletion of one, two, three, five, ten or more nucleotides, of polynucleotides encoding the delivery construct. Alternatively, derivatives can be made by random mutagenesis. One method for randomly mutagenizing a nucleic acid comprises amplifying the nucleic acid in a PCR reaction in the presence of 0.1 mM MnCl₂ and unbalanced nucleotide concentrations. These conditions increase the misincorporation rate of the polymerase used in the PCR reaction and result in random mutagenesis of the amplified nucleic acid.

Several site-specific mutations and deletions in chimeric molecules derived from PE have been made and characterized. For example, deletion of nucleotides encoding amino acids 1-252 of PE yields a construct referred to as “PE40-” Deleting nucleotides encoding amino acids 1-279 of PE yields a construct referred to as “PE37.” See U.S. Pat. No. 5,602,095. In both of these constructs, the receptor binding domain of PE, i.e., domain Ia, has been deleted. Nucleic acids encoding a receptor binding domain can be ligated to these constructs to produce delivery constructs that are targeted to the cell surface receptor recognized by the receptor binding domain. Of course, these recombinant polynucleotides are particularly useful for expressing delivery constructs that have a receptor binding domain that is not domain Ia of PE. The recombinant polynucleotides can optionally encode an amino-terminal methionine to assist in expression of the construct. In certain embodiments, the receptor binding domain can be ligated to the 5′ end of the polynucleotide encoding the transcytosis domain.

Other nucleic acids encoding mutant forms of PE that can be used as a source of nucleic acids for constructing the delivery constructs of the invention include, but are not limited to, PEA553 and those described in U.S. Pat. Nos. 5,602,095; 5,512,658 and 5,458,878, and in Vasil et al., 1986, Infect. Immunol. 52:538-48.

Accordingly, in certain embodiments, the invention provides a polynucleotide that encodes a delivery construct. The delivery construct comprises a receptor binding domain, a transcytosis domain, a GH to be delivered to a subject, and a cleavable linker. Cleavage at the cleavable linker can separate the GH from the remainder of the construct. The cleavable linker can be cleaved by an enzyme that is present at a basal-lateral membrane of a polarized epithelial cell of the subject or in the plasma of the subject.

In certain embodiments, the polynucleotide hybridizes under stringent hybridization conditions to any polynucleotide of this invention. In further embodiments, the polynucleotide hybridizes under stringent conditions to a nucleic acid that encodes any delivery construct of the invention.

In certain embodiments, the polynucleotide encodes a delivery construct that further comprises a second cleavable linker. In certain embodiments, the first and/or second cleavable linker comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7). In certain embodiments, the first and/or second cleavable linker encoded by the polynucleotide is cleavable by an enzyme that is selected from the group consisting of Cathepsin GI, Chymotrypsin I, Elastase I, Subtilisin AI, Subtilisin AII, Thrombin I, and Urokinase I.

In certain embodiments, the receptor binding domain encoded by the polynucleotide is selected from the group consisting of receptor binding domains from Pseudomonas exotoxin A, cholera toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain encoded by the polynucleotide binds to a cell-surface receptor that is selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor. In further embodiments, the receptor binding domain encoded by the polynucleotide is Domain Ia of Pseudomonas exotoxin A. In yet further embodiments, the receptor binding domain encoded by the polynucleotide has an amino acid sequence that is SEQ ID NO.:9.

In certain embodiments, the transcytosis domain encoded by the polynucleotide is selected from the group consisting of transcytosis domains from Pseudomonas exotoxin A, diptheria toxin, pertussis toxin, cholera toxin, heat-labile E. coli enterotoxin, shiga toxin, and shiga-like toxin. In further embodiments, the transcytosis domain is Pseudomonas exotoxin A transcytosis domain. In still further embodiments, the Pseudomonas exotoxin A transcytosis domain has an amino acid sequence that is SEQ ID NO.:10.

In certain embodiments, the GH is human growth hormone.

5.6. Expression Vectors

In still another aspect, the invention provides expression vectors for expressing the delivery constructs. Generally, expression vectors are recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation of mRNA. Techniques for construction of expression vectors and expression of genes in cells comprising the expression vectors are well known in the art. See, e.g., Sambrook et al., 2001, Molecular Cloning—A Laboratory Manual, 3^{rd} edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

Useful promoters for use in expression vectors include, but are not limited to, a metallothionein promoter, a constitutive adenovirus major late promoter, a dexamethasone-inducible MMTV promoter, a SV40 promoter, a MRP pol III promoter, a constitutive MPSV promoter, a tetracycline-inducible CMV promoter (such as the human immediate-early CMV promoter), and a constitutive CMV promoter.

The expression vectors should contain expression and replication signals compatible with the cell in which the delivery constructs are expressed. Expression vectors useful for expressing delivery constructs include viral vectors such as retroviruses, adenoviruses and adenoassociated viruses, plasmid vectors, cosmids, and the like. Viral and plasmid vectors are preferred for transfecting the expression vectors into mammalian cells. For example, the expression vector pcDNA1 (Invitrogen, San Diego, Calif.), in which the expression control sequence comprises the CMV promoter, provides good rates of transfection and expression into such cells.

The expression vectors can be introduced into the cell for expression of the delivery constructs by any method known to one of skill in the art without limitation. Such methods include, but are not limited to, e.g., direct uptake of the molecule by a cell from solution; facilitated uptake through lipofection using, e.g., liposomes or immunoliposomes; particle-mediated transfection; etc. See, e.g., U.S. Pat. No. 5,272,065; Goeddel et al., eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, N.Y.; and Ausubel et al., eds., Current Edition, Current Protocols in Molecular Biology, Greene Publishing Associates and Wiley Interscience, NY.

The expression vectors can also contain a purification moiety that simplifies isolation of the delivery construct. For example, a polyhistidine moiety of, e.g., six histidine residues, can be incorporated at the amino terminal end of the protein. The polyhistidine moiety allows convenient isolation of the protein in a single step by nickel-chelate chromatography. In certain embodiments, the purification moiety can be cleaved from the remainder of the delivery construct following purification. In other embodiments, the moiety does not interfere with the function of the functional domains of the delivery construct and thus need not be cleaved.

5.7. Cell for Expressing a Delivery Construct

In yet another aspect, the invention provides a cell comprising an expression vector for expression of the delivery constructs, or portions thereof. The cell is preferably selected for its ability to express high concentrations of the delivery construct to facilitate purification of the protein. In certain embodiments, the cell is a prokaryotic cell, for example, E. coli. As described in the examples, the delivery constructs are properly folded and comprise the appropriate disulfide linkages when expressed in E. coli.

In other embodiments, the cell is a eukaryotic cell. Useful eukaryotic cells include yeast and mammalian cells. Any mammalian cell known by one of skill in the art to be useful for expressing a recombinant polypeptide, without limitation, can be used to express the delivery constructs. For example, Chinese hamster ovary (CHO) cells can be used to express the delivery constructs.

5.8. Compositions Comprising Delivery Constructs

The delivery constructs of the invention can be formulated as compositions. The compositions are generally formulated appropriately for the immediate use intended for the delivery construct. For example, if the delivery construct is not to be administered immediately, the delivery construct can be formulated in a composition suitable for storage. One such composition is a lyophilized preparation of the delivery construct together with a suitable stabilizer. Alternatively, the delivery construct composition can be formulated for storage in a solution with one or more suitable stabilizers. Any such stabilizer known to one of skill in the art without limitation can be used. For example, stabilizers suitable for lyophilized preparations include, but are not limited to, sugars, salts, surfactants, proteins, chaotropic agents, lipids, and amino acids. Stabilizers suitable for liquid preparations include, but are not limited to, sugars, salts, surfactants, proteins, chaotropic agents, lipids, and amino acids. Specific stabilizers than can be used in the compositions include, but are not limited to, trehalose, serum albumin, phosphatidylcholine, lecithin, and arginine. Other compounds, compositions, and methods for stabilizing a lyophilized or liquid preparation of the delivery constructs may be found, for example, in U.S. Pat. Nos. 6,573,237, 6,525,102, 6,391,296, 6,255,284, 6,133,229, 6,007,791, 5,997,856, and 5,917,021.

Further, the delivery construct compositions of the invention can be formulated for administration to a subject. Such vaccine compositions generally comprise one or more delivery constructs of the invention and a pharmaceutically acceptable excipient, diluent, carrier, or vehicle. Any such pharmaceutically acceptable excipient, diluent, carrier, or vehicle known to one of skill in the art without limitation can be used. Examples of a suitable excipient, diluent, carrier, or vehicle can be found in Remington's Pharmaceutical Sciences, 21st Ed. 2005, Mack Publishing Co., Easton.

In certain embodiments, the delivery construct compositions are formulated for oral administration. In such embodiments, the compositions are formulated to protect the delivery construct from acid and/or enzymatic degradation in the stomach. Upon passage to the neutral to alkaline environment of the duodenum, the delivery construct then contacts a mucous membrane and is transported across the polarized epithelial membrane. The delivery constructs may be formulated in such compositions by any method known by one of skill in the art, without limitation.

In certain embodiments, the oral formulation comprises a delivery construct and one or more compounds that can protect the delivery construct while it is in the stomach. For example, the protective compound should be able to prevent acid and/or enzymatic hydrolysis of the delivery construct. In certain embodiments, the oral formulation comprises a delivery construct and one or more compounds that can facilitate transit of the construct from the stomach to the small intestine. In certain embodiments, the one or more compounds that can protect the delivery construct from degradation in the stomach can also facilitate transit of the construct from the stomach to the small intestine. Preferably, the oral formulation comprises one or more compounds that can protect the delivery construct from degradation in the stomach and facilitate transit of the construct from the stomach to the small intestine. For example, inclusion of sodium bicarbonate can be useful in facilitating the rapid movement of intra-gastric delivered materials from the stomach to the duodenum as described in Mrsny et al., 1999, Vaccine 17:1425-1433.

Other methods for formulating compositions so that the delivery constructs can pass through the stomach and contact polarized epithelial membranes in the small intestine include, but are not limited to, enteric-coating technologies as described in DeYoung, 1989, Int J Pancreatol. 5 Suppl:31-6, and the methods provided in U.S. Pat. Nos. 6,613,332, 6,174,529, 6,086,918, 5,922,680, and 5,807,832.

5.8.1. Kits Comprising Compositions

In yet another aspect, the invention provides a kit that comprises a composition of the invention. In certain embodiments, the kit further comprises instructions that direct administration of the composition to a mucous membrane of the subject to whom the composition is administered. In certain embodiments, the kit further comprises instructions that direct oral administration of the composition to the subject to whom the composition is administered.

In certain embodiments, the kit comprises a composition of the invention in more or more containers. In certain embodiments, the composition can be in a unit dosage form, e.g., a tablet, lozenge, capsule, etc. In certain embodiments, the composition can be provided in or with a device for administering the composition, such as, for example, a device configured to administer a single-unit dose of the composition, e.g., an inhaler.

5.9. Making and Testing Delivery Constructs

The delivery constructs of the invention are preferably produced recombinantly, as described below. However, the delivery constructs may also be produced by chemical synthesis using methods known to those of skill in the art.

5.9.1. Manufacture of Delivery Constructs

Methods for expressing and purifying the delivery constructs of the invention are described extensively in the examples below. Generally, the methods rely on introduction of an expression vector encoding the delivery construct to a cell that can express the delivery construct from the vector. The delivery construct can then be purified for administration to a subject.

5.9.2. Testing Delivery Constructs

Having selected the domains of the delivery construct, the function of these domains, and of the delivery constructs as a whole, can be routinely tested to ensure that the constructs can deliver a GH across mucous membranes of a subject free from the remainder of the construct. For example, the delivery constructs can be tested for cell recognition, transcytosis and cleavage using routine assays. The entire chimeric protein can be tested, or, the function of various domains can be tested by substituting them for native domains of the wild-type toxin.

5.9.2.1. Receptor Binding/Cell Recognition

Receptor binding domain function can be tested by monitoring the delivery construct's ability to bind to the target receptor. Such testing can be accomplished using cell-based assays, with the target receptor present on a cell surface, or in cell-free assays. For example, delivery construct binding to a target can be assessed with affinity chromatography. The construct can be attached to a matrix in an affinity column, and binding of the receptor to the matrix detected, or vice versa. Alternatively, if antibodies have been identified that bind to either the receptor binding domain or its cognate receptor, the antibodies can be used, for example, to detect the receptor binding domain in the delivery construct by immunoassay, or in a competition assay for the cognate receptor. An exemplary cell-based assay that detects delivery construct binding to receptors on cells comprises labeling the construct and detecting its binding to cells by, e.g., fluorescent cell sorting, autoradiography, etc.

5.9.2.2. Transcytosis

The function of the transcytosis domain can be tested as a function of the delivery construct's ability to pass through an epithelial membrane. Because transcytosis first requires binding to the cell, these assays can also be used to assess the function of the cell recognition domain.

The delivery construct's transcytosis activity can be tested by any method known by one of skill in the art, without limitation. In certain embodiments, transcytosis activity can be tested by assessing the ability of a delivery construct to enter a non-polarized cell to which it binds. Without intending to be bound to any particular theory or mechanism of action, it is believed that the same property that allows a transcytosis domain to pass through a polarized epithelial cell also allows molecules bearing the transcytosis domain to enter non-polarized cells. Thus, the delivery construct's ability to enter the cell can be assessed, for example, by detecting the physical presence of the construct in the interior of the cell. For example, the delivery construct can be labeled with, for example, a fluorescent marker, and the delivery construct exposed to the cell. Then, the cells can be washed, removing any delivery construct that has not entered the cell, and the amount of label remaining determined. Detecting the label in this fraction indicates that the delivery construct has entered the cell.

In other embodiments, the delivery construct's transcytosis ability can be tested by assessing the delivery construct's ability to pass through a polarized epithelial cell. For example, the delivery construct can be labeled with, for example, a fluorescent marker and contacted to the apical membranes of a layer of epithelial cells. Fluorescence detected on the basal-lateral side of the membrane formed by the epithelial cells indicates that the transcytosis domain is functioning properly.

5.9.2.3. Cleavable Linker Cleavage

The function of the cleavable linker can generally be tested in a cleavage assay. Any suitable cleavage assay known by one of skill in the art, without limitation, can be used to test the cleavable linkers. Both cell-based and cell-free assays can be used to test the ability of an enzyme to cleave the cleavable linkers.

An exemplary cell-free assay for testing cleavage of cleavable linkers comprises preparing extracts of polarized epithelial cells and exposing a labeled delivery construct bearing a cleavable linker to the fraction of the extract that corresponds to membrane-associated enzymes. In such assays, the label can be attached to either the GH to be delivered or to the remainder of the delivery construct. Among these enzymes are cleavage enzymes found near the basal-lateral membrane of a polarized epithelial cell, as described above. Cleavage can be detected, for example, by binding the delivery construct with, for example, an antibody and washing off unbound molecules. If label is attached to the GH to be delivered, then little or no label should be observed on the molecule bound to the antibodies. Alternatively, the binding agent used in the assay can be specific for the GH, and the remainder of the construct can be labeled. In either case, cleavage can be assessed.

Cleavage can also be tested using cell-based assays that test cleavage by polarized epithelial cells assembled into membranes. For example, a labeled delivery construct, or portion of a delivery construct comprising the cleavable linker, can be contacted to either the apical or basolateral side of a monolayer of suitable epithelial cells, such as, for example, Coco-2 cells, under conditions that permit cleavage of the linker. Cleavage can be detected by detecting the presence or absence of the label using a reagent that specifically binds the delivery construct, or portion thereof. For example, an antibody specific for the delivery construct can be used to bind a delivery construct comprising a label distal to the cleavable linker in relation to the portion of the delivery construct bound by the antibody. Cleavage can then be assessed by detecting the presence of the label on molecules bound to the antibody. If cleavage has occurred, little or no label should be observed on the molecules bound to the antibody. By performing such experiments, enzymes that preferentially cleave at the basolateral membrane rather than the apical membrane can be identified, and, further, the ability of such enzymes to cleave the cleavable linker in a delivery construct can be confirmed.

Further, cleavage can also be tested using a fluorescence reporter assay as described in U.S. Pat. No. 6,759,207. Briefly, in such assays, the fluorescence reporter is contacted to the basolateral side of a monolayer of suitable epithelial cells under conditions that allow the cleaving enzyme to cleave the reporter. Cleavage of the reporter changes the structure of the fluorescence reporter, changing it from a non-fluorescent configuration to a fluorescent configuration. The amount of fluorescence observed indicates the activity of the cleaving enzyme present at the basolateral membrane.

Further, cleavage can also be tested using an intra-molecularly quenched molecular probe, such as those described in U.S. Pat. No. 6,592,847. Such probes generally comprise a fluorescent moiety that emits photons when excited with light of appropriate wavelength and a quencher moiety that absorbs such photons when in close proximity to the fluorescent moiety. Cleavage of the probe separates the quenching moiety from the fluorescent moiety, such that fluorescence can be detected, thereby indicating that cleavage has occurred. Thus, such probes can be used to identify and assess cleavage by particular cleaving enzymes by contacting the basolateral side of a monolayer of suitable epithelial cells with the probe under conditions that allow the cleaving enzyme to cleave the probe. The amount of fluorescence observed indicates the activity of the cleaving enzyme being tested.

### 6. EXAMPLES

The following examples merely illustrate the invention, and are not intended to limit the invention in any way.

6.1. Construction of a Delivery Construct

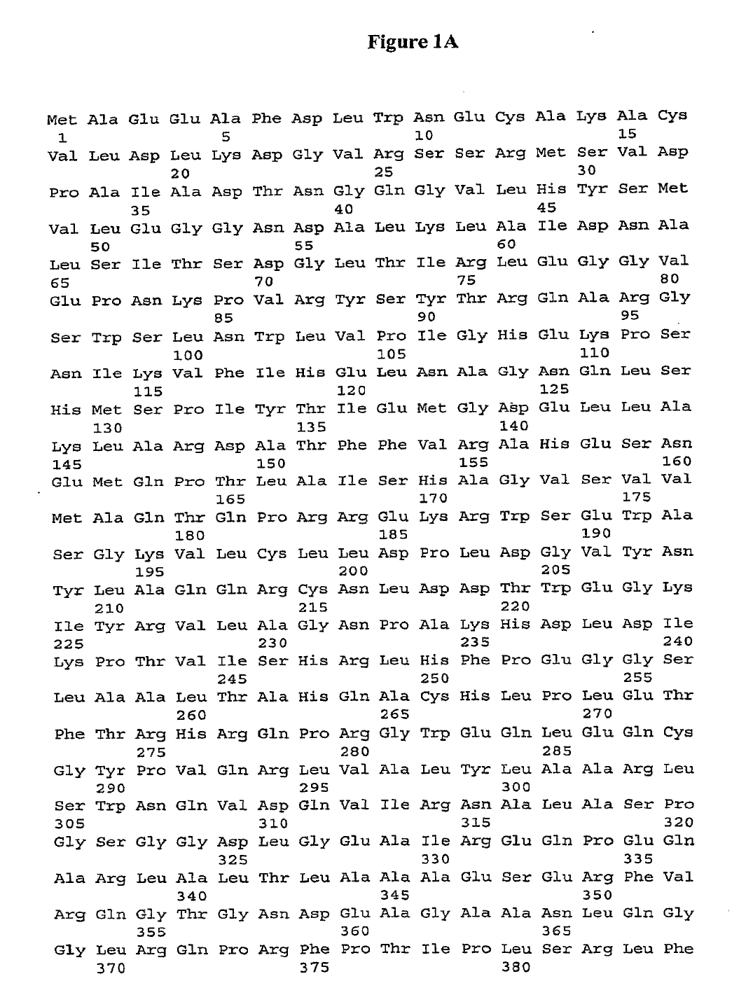

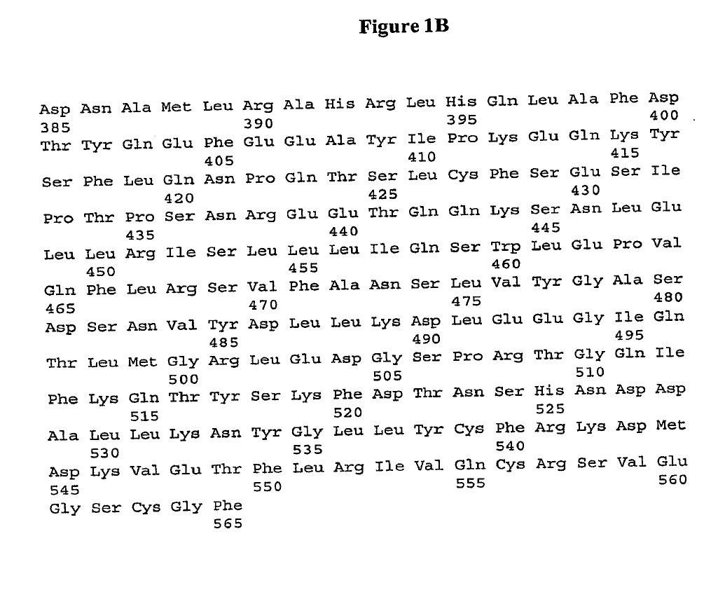

An exemplary delivery construct expression vector for delivering human growth hormone (hGH) was constructed according to the following protocol. First, the hGH gene was amplified by PCR, incorporating restriction enzymes recognition sites at two ends of the PCR products. After restriction enzyme digestion, the PCR products were cloned into pPE64-PstI-Δ553, which was digested with the corresponding restriction enzyme pairs. These constructs thus comprise sequences encoding Domains I and II of ntPE (amino acids 26-372 as shown in FIG. 1) and hGH (Accession No. AAA72260; see Ikehara et al, 1984, Proc. Natl. Acad. Sci. U.S.A. 81:5956-5960), and are also tagged with a 6-His motif at the N-terminus of the polypeptide to facilitate purification. The final plasmids were verified by restriction enzyme digestions and DNA sequencing.

Next, an expression vector for expressing a delivery construct comprising a cleavable linker between the hGH portion of the delivery construct and the remainder of the molecule was constructed. For this exemplary delivery construct, referred to herein as “HGH Delivery Construct,” the cleavable linker sequence introduced was GGLRQPR. To do so, oligonucleotides that encode the specified amino acid sequence flanked by appropriate restriction sites and one of the following amino acid sequences were synthesized, then ligated into an expression vector prepared as described above between the ntPE sequences and the hGH sequences.

To separate hGH from remainder of the molecule in the event, for example, that the fusion protein is taken up by antigen presenting cells, a protease furin site was also inserted between the cleavable linker and rGH. To do so, constructs containing a sequence encoding the furin site in combination with the cleavable linker was made. Oligonucleotide sequences for the five cleavable linkers and a furin clip site are shown in Table 3, below. The final construct was confirmed by restriction enzyme digestion and DNA sequencing.

TABLE 3
Oligonucleotides Encoding Cleavable Linkers

AACTGCAGGGAGGCTTACGCCAGCCTCGACTGCAGAA
(SEQ ID NO: 25)
TTCTGCAGTCGAGGCTGGCGTAAGCCTCCCTGCAGTT
(SEQ ID NO: 26)

6.2. Expression of Delivery Constructs

E. coli BL21(DE3) pLysS competent cells (Novagen, Madison, Wis.) were transformed using a standard heat-shock method in the presence of the appropriate plasmid to generate ntPE-human Growth Hormone (hGH) expression cells, selected on ampicillin-containing media, and isolated and grown in Luria-Bertani broth (Difco; Becton Dickinson, Franklin Lakes, N.J.) with antibiotic, then induced for protein expression by the addition of 1 mM isopropyl-D-thiogalactopyranoside (IPTG) at OD 0.6. Two hours following IPTG induction, cells were harvested by centrifugation at 5,000 rpm for 10 min. Inclusion bodies were isolated following cell lysis and proteins were solubilized in the buffer containing 100 mM Tris-HCl (pH 8.0), 2 mM EDTA, 6 M guanidine HCl, and 65 mM dithiothreitol. Solubilized His ntPE-rGH is refolded in the presence of 0.1 M Tris, pH=7.4, 500 mM L-arginine, 0.9 mM GSSG, 2 mM EDTA. The refolded proteins were purified by Q sepharose Ion Exchange and Superdex 200 Gel Filtration chromatography (Amersham BioSciences, Inc., Sweden). The purity of proteins was assessed by SDS-PAGE and analytic HPLC (Agilent, Inc. Palo Alto, Calif.).

6.3. Characterization of a Delivery Construct

One or more of the following procedures are used to assess proper refolding of a delivery construct. The protein refolding process is monitored by measuring, e.g., Delivery Construct 1 binding activity with ntPE binding receptor, CD 91 receptors, and hGH binding proteins on a Biacore SPR instrument (Biacore, Sweden) according to the manufacturer's instructions.

6.4. Detection of Growth Hormone Protein in Tissue by Histological Examination

This example describes histological detection in tissues of a representative GH for delivery, human growth hormone. Following administration of a delivery construct, animals are euthanized by CO₂ asphyxiation and exanguinated by cardiac puncture. Specific tissues (lymph nodes, trachea, brain, spleen liver, GI tract) are removed, briefly rinsed in PBS to remove any residual blood and frozen in OCT. Sections (5 microns thick) are placed onto slides. Slides are fixed in acetone for 10 min and rinsed with PBS. Slides are incubated with 3% peroxidase for 5 min. Slides are then blocked with protein for an additional 5 min. Primary anti-human growth hormone antibody is incubated onto slides for 30 min at a 1:100 dilution followed by PBS washes. Biotin-labeled secondary antibody is then incubated for approximately 15 minutes followed by PBS washes. Streptavidin HRP label is incubated onto slides for 15 min followed by PBS washes. HRP Chromagen is applied for 5 min followed by several rinses in distilled H₂O. Finally, the slides are counterstained with hematoxylin for 1 min, coverslipped, and examined for the presence of GH.

6.5. Delivery of an Exemplary Growth Hormone in an In Vivo System

This example describes use of exemplary HGH Delivery Construct in a mouse model, showing effective transport and cleavage of the delivery construct in vivo, the bioactivity of the GH delivered by HGH Delivery Construct, hGH, and the effects of hGH on mouse growth.

6.5.1. Administration of a Delivery Construct Comprising Rat Growth Hormone

Female mice of a genetically growth-deficient strain known as Little (lit/lit) or Lit=C57BL/6J-Ghrhr<lit>/J (The Jackson Laboratory, Bar Harbor, Me.) were weighed for three consecutive days to establish a stable baseline weight (verifying growth restriction as well as general health) and randomly sorted to a treatment group. Treatment groups included mice administered an amount of HGH Delivery Construct corresponding to 30 μg or 60 μg hGH, mice administered 30 μg or 60 μg recombinant hGH (rhGH), and mice administered either PBS subcutaneously or left untreated as a negative control.

The HGH Delivery Construct was administered as follows. Mice were dosed intranasally by diluting the HGH Delivery Construct in phosphate buffered saline (pH 7.4) to the proper concentration to deliver a desired dose in 40 μl (20 μl/nares) using a positive displacement pipette. Dosing was performed under mild anesthesia induced by isoflurane.

Mice administered rhGH received subcutaneous injections of rhGH (QED Bioscience, Inc.; San Diego, Calif.) in an injection volume of 100 μl following reconstitution of the lyophilized preparation with water according to the manufacturer's instructions. Dosing was performed under mild anesthesia induced by isoflurane.

In addition, to compare the pharmacokinetic and pharmacodynamic properties of oral and intranasal administration of HGH Delivery Construct, HGH Delivery Construct was also orally administered to lit/lit mice according to the following procedure. An amount of HGH Delivery Construct corresponding to either 30 μg or 300 μg hGH (in 250 μl total volume) was administered orally using an animal feeding needle to mice. The HGH Delivery Construct was diluted in 1 mg/ml bovine serum albumin (BSA) and phosphate buffered saline (PBS) prior to administration.

All groups (oral, intranasal, and subcutaneous administration) were dosed daily and weighed daily for ten days. All weights were determined using a scale calibrated just prior to each weighing session. On day 10 of administration, mice from all groups were euthanized by CO₂ asphyxiation and exsanguinated 30 minutes after the final administration of hGH. Serum concentrations of hGH, bioactive hGH, IGF-1, IGF1-BP3, anti-hGH IgG antibodies, anti-ntPE IgG antibodies, corticosterone, leptin, and insulin were determined using commercial ELISA assays for mice from each group.

6.5.2. Growth of Mice Administered HGH Delivery Construct

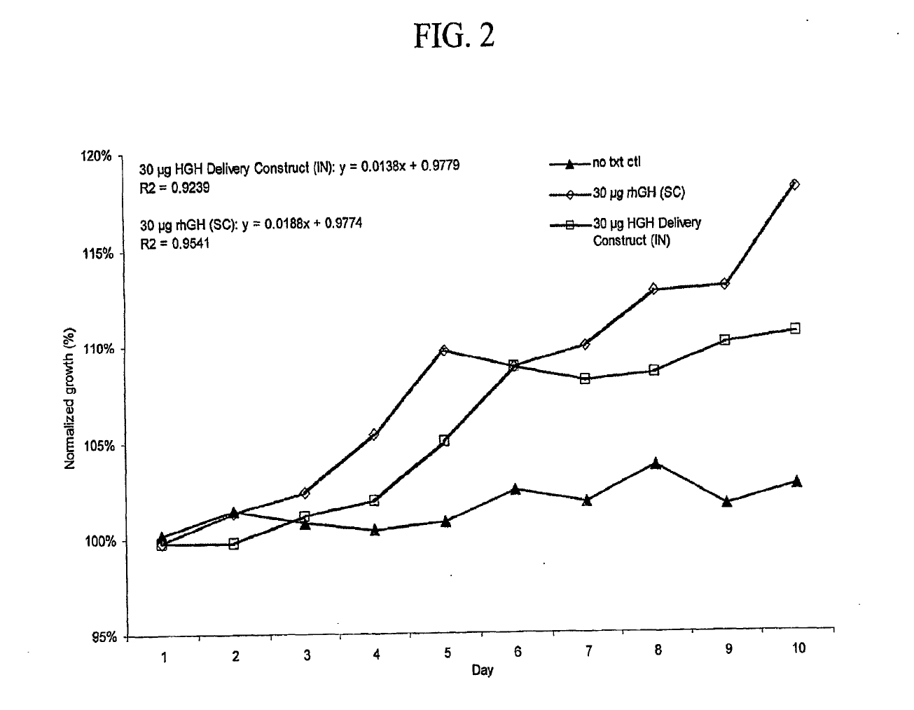

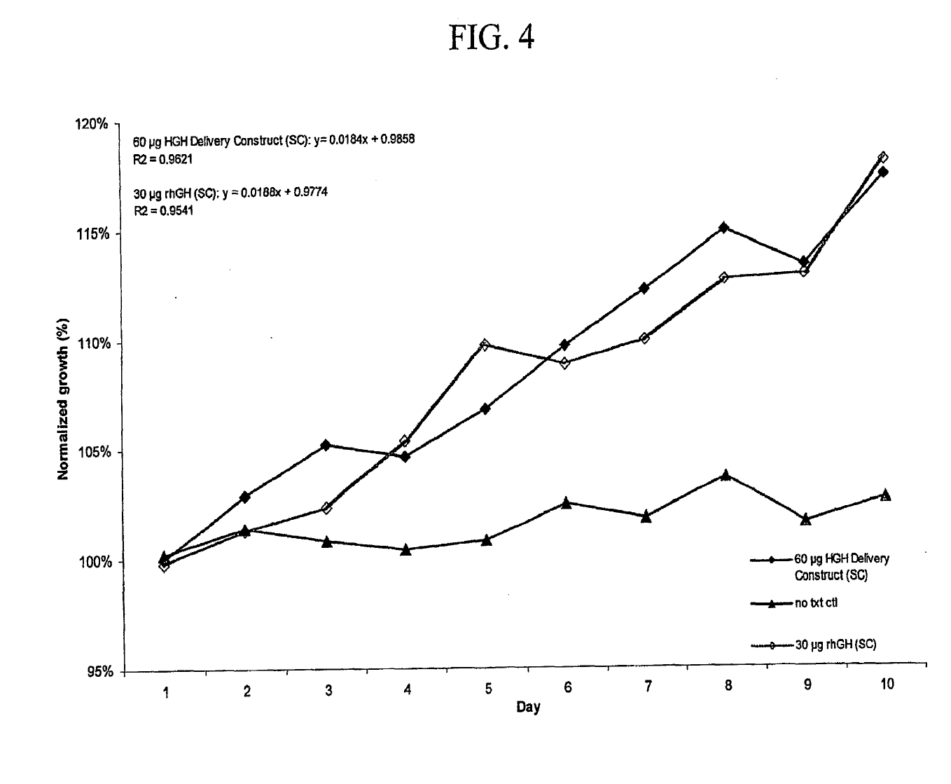

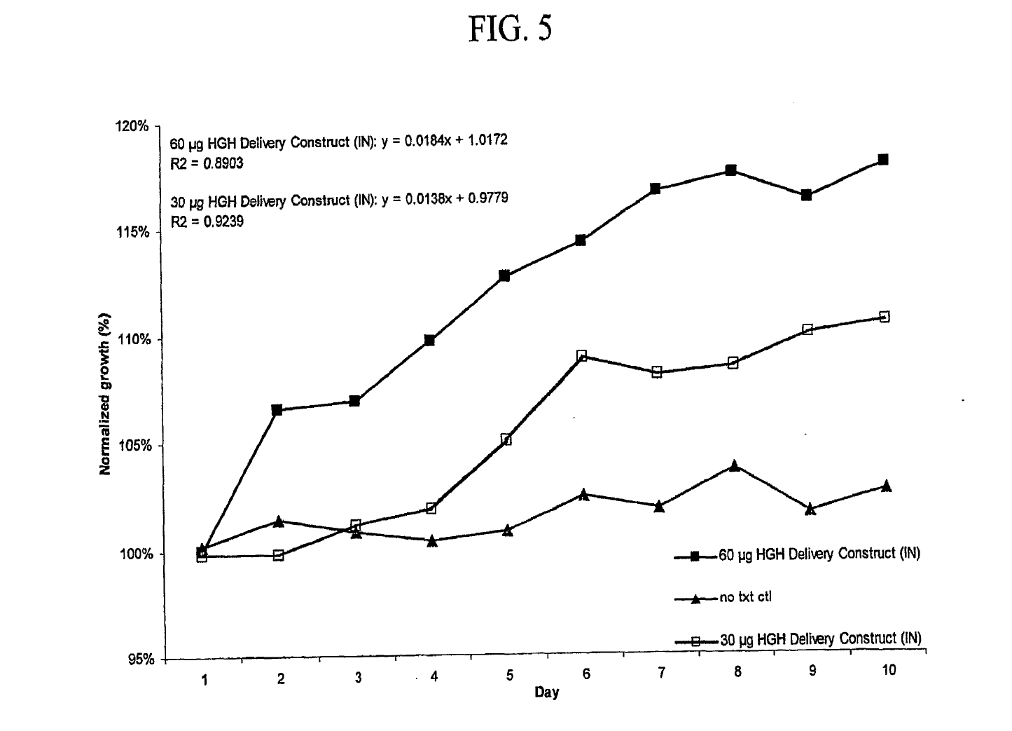

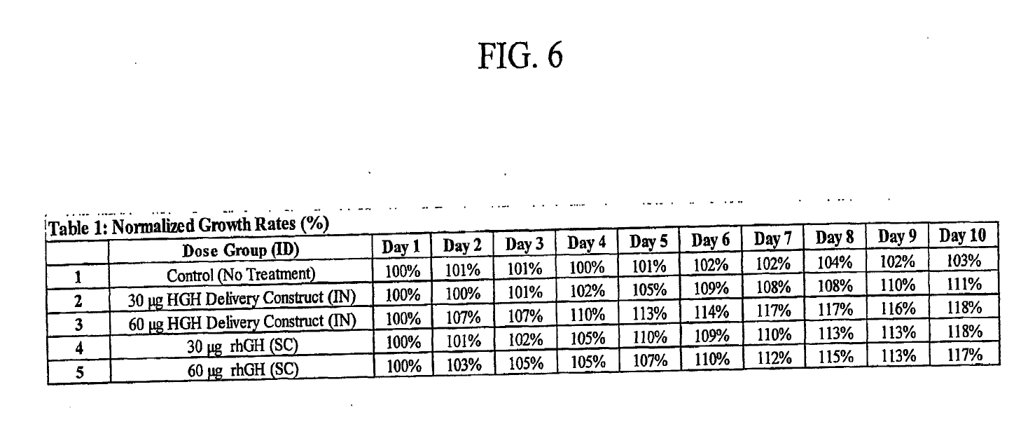

The results of the growth experiments conducted as described in Section 6.5.1, above, are presented in FIGS. 2-6. FIG. 2 compares the weight gain observed for mice administered 30 μg hGH subcutaneously or 30 μg hGH intranasally with the HGH Delivery Construct. FIG. 3 compares the weight gain observed for mice administered 60 μg hGH subcutaneously or 60 μg hGH intranasally with the HGH Delivery Construct. FIG. 4 compares the weight gain observed for mice administered either 30 μg hGH or 60 μg hGH subcutaneously. FIG. 5 compares the weight gain observed for mice administered either 30 μg hGH or 60 μg hGH intranasally with the HGH Delivery Construct. FIG. 6 presents a table of the underlying data used to construct the graphs of FIGS. 2-5. No data are presented for mice administered the HGH Delivery Construct orally as such mice did not significantly increase in weight. As shown in FIGS. 5 and 6, administration of 60 μg hGH intranasally with the HGH Delivery Construct resulted in a weight gain of 13% by day 5 of administration and a weight gain of 18% by day 10 of administration.

The slopes of the best-fit lines were calculated for each of the different experimental groups shown in FIGS. 2-5 and used to calculate the effects of the hGH administered on growth rate. Results of these calculations are shown in Table 4, below.

TABLE 4

| | Effect on Growth Rate |
|---|---|
| | (% weight change/ |
| Amount and Route of hGH Administration | day · μg hGH) |
| 30 μg hGH IN with HGH Delivery Construct | 4.6 × 10⁻⁴ |
| 60 μg hGH IN with HGH Delivery Construct | 4.8 × 10⁻⁴ |
| 30 μg hGH DC | 6.5 × 10⁻⁴ |
| 60 μg hGH DC | 3.1 × 10⁻⁴ |

As shown in Table 4, administration of 30 μg hGH and 60 μg hGH intranasally with the HGH Delivery Construct resulted in essentially identical effects on the rate of growth of the mice. In contrast, 60 μg rhGH administered subcutaneously did not increase the rate of growth of the mice beyond that observed for mice administered 30 μg rhGH subcutaneously. Thus, these experiments demonstrate that a higher effective dose of hGH can be effectively administered intranasally with the HGH Delivery Construct than can be administered subcutaneously.

6.5.3. Pharmacokinetics of an Exemplary Growth Hormone Administered with a Delivery Construct in an In Vivo System

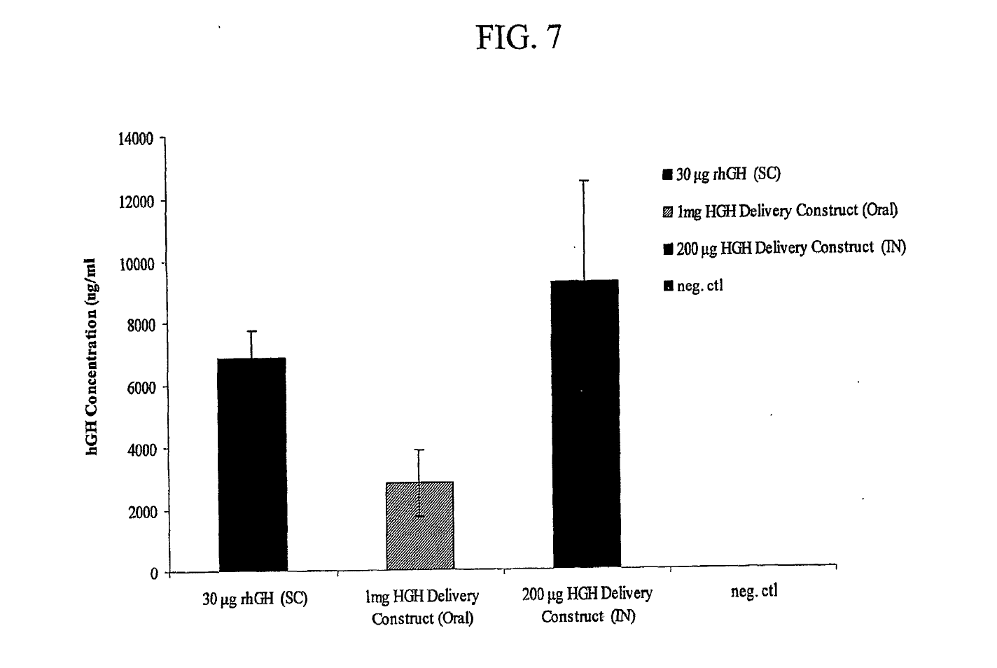

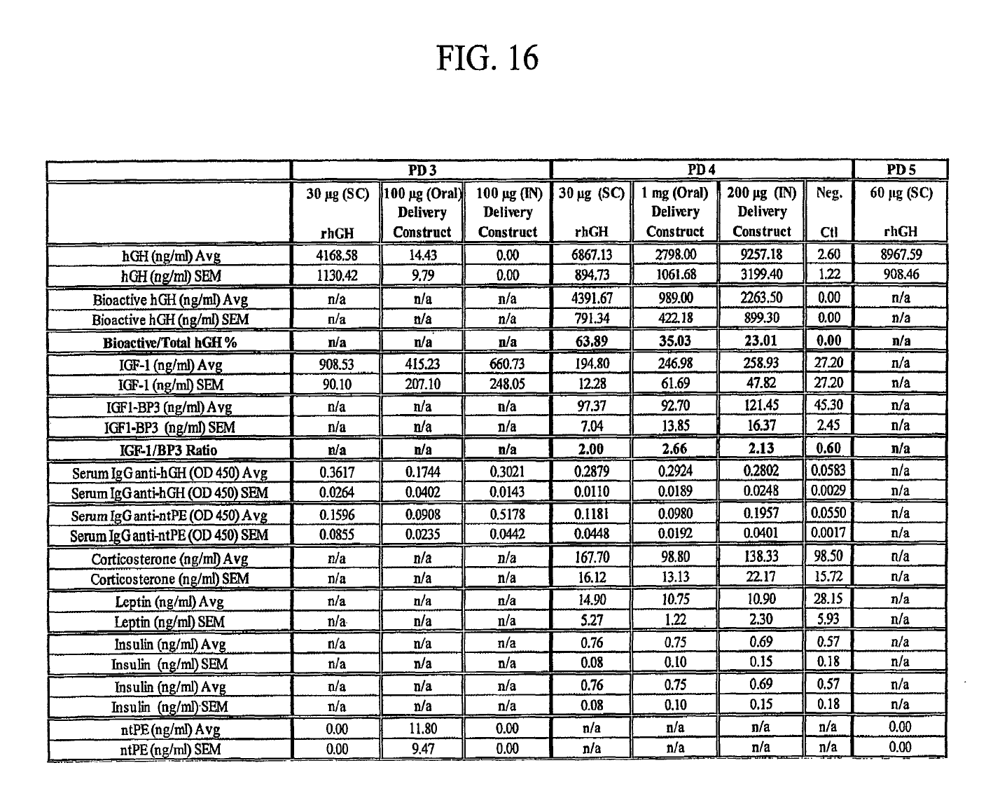

To assess effects of administration of rhGH administered subcutaneously and hGH administered orally or intranasally with HGH Delivery Construct, serum concentrations of several molecules were determined with ELISA assays. In particular, serum concentrations of hGH, bioactive hGH, IGF-1, IGF1-BP3, anti-hGH IgG antibodies, anti-ntPE IgG antibodies, corticosterone, leptin, insulin, and ntPE were measured in lit/lit mouse serum 30 minutes following the day 10 administration of hGH. Serum concentrations of at least some of these molecules were determined for 30 μg hGH administered subcutaneously, 30 μg hGH administered orally with the HGH Delivery Construct, 30 μg hGH administered intranasally with the HGH delivery construct, 300 μg hGH administered orally with the HGH Delivery Construct, 60 μg hGH administered intranasally with the HGH delivery construct, and 60 μg hGH administered subcutaneously. Data thus obtained is presented in FIGS. 7-15 and summarized in the Table presented as FIG. 16.

Serum concentrations of hGH, bioactive hGH, IGF-1, IGF1-BP3, corticosterone, leptin, and insulin were determined using commercially available kits for performing ELISA assays. The exact kit, its supplier, and the molecule measured with the kit are presented in Table 5, below.

TABLE 5

| Molecule | Supplier | Catalog # or Protocol # |
|---|---|---|
| hGH | Diagnostic Systems Laboratories | DSL-10-19100, Ultra-Sensitive Human |
| | (Webster, TX) | Growth Hormone ELISA |
| Bioactive hGH | Diagnostic Systems Laboratories | DSL-10-11100, Bioactive GH ELISA |
| | (Webster, TX) | |
| IGF-1 | R & D Systems | MG100, Mouse IGF-1 Immunoassay |
| | (Minneapolis, MN) | |
| IGF-1 BP3 | R & D Systems | DY775, Mouse IGFBP-3 ELISA |
| | (Minneapolis, MN) | Development System |
| Corticosterone | Neogen Corporation | Product # 402810, Corticosterone ELISA |
| | (Lansing, MI) | |
| Leptin | R & D Systems | MOB00, Mouse Leptin Immunoassay |
| | (Minneapolis, MN) | |
| Insulin | ALPCO Diagnostics | 10-1150-01, Mercodia Ultrasensitive |
| | (Salem, NH) | Mouse Insulin ELISA |

ELISA assays to determine the concentration of anti-hGH IgG antibodies, anti-ntPE IgG antibodies, and ntPE were performed as follows:

To measure mouse anti-hGH IgG antibodies, Costar 9018 E.I.A./R.I.A. 96-well plates were coated overnight with 100 ng/well of rhGH (QED, Cat. No. 20901) in 0.2M NaHCO₃—Na₂CO₃, pH 9.4. Each 96-well plate was washed four times with PBS containing 0.05% Tween 20-0.01% thimerosal (wash buffer); blocked for 1 h with 200 μl/well of PBS/Tween 20 containing 0.5% BSA-0.01% thimerosal (assay buffer). Each plate was washed again and serum samples were diluted at 1:20 in assay buffer. Samples were loaded in 100 μl/well triplicates onto a 96-well plate, and incubated 1 h to detect specific mouse serum IgG. Each 96-well plate was then washed four times with wash buffer, and added 100 μl/well of horseradish peroxidase (HRP) conjugated goat anti-mouse IgG (Pierce, Cat. No.31430) at 1:6000 dilutions and incubated for 1 h. All incubation and coating steps were performed at room temperature on a shaker at 6 RPM. The HRP substrate, TMB (3,3′,5,5′tetramethylbenzidine), used to quantify bound antibody, was measured at 450 nm.

To measure mouse anti-ntPE IgG antibodies, Costar 9018 E.I.A./R.I.A. 96-well plates were coated overnight with 200 ng/well of ntPE in 0.2M NaHCO₃—Na₂CO₃, pH 9.4. Each 96-well plate was washed four times with PBS containing 0.05% Tween 20-0.01% thimerosal (wash buffer); blocked for 1 h with 200 μl/well of PBS/Tween 20 containing 0.5% BSA-0.01% thimerosal (assay buffer). Each plate was washed again and serum samples were diluted at 1:20 in assay buffer. Samples were loaded in 100 μl/well triplicates onto a 96-well plate, and incubated 1 h to detect specific mouse serum IgG. Each 96-well plate was then washed four times with wash buffer, and added 100 μl/well of horseradish peroxidase (HRP) conjugated goat anti-mouse IgG (Pierce, Cat. No.31430) at 1:6000 dilutions and incubated for 1 h. All incubation and coating steps were performed at room temperature on a shaker at 6 RPM. The HRP substrate, TMB (3,3′,5,5′tetramethylbenzidine), used to quantify bound antibody, was measured at 450 nm.

To measure ntPE concentrations in serum samples, Costar 9018 E.I.A./R.I.A. 96-well plates were coated overnight with 200 ng/well of M40-1 mAb (specific for ntPE) in 0.2M NaHCO₃—Na₂CO₃, pH 9.4. Each 96-well plate was washed four times with PBS containing 0.05% Tween 20-0.01% thimerosal (wash buffer); blocked for 1 h with 200 μl/well of PBS/Tween 20 containing 0.5% BSA-0.01% thimerosal (assay buffer). Purified ntPE diluted in assay buffer was used as the standard curve. Standard curve was prepared by adding 5 μl of the 10 mg/ml ntPE to 10 ml assay buffer (1:2000), mixing well and moving 50 μl to 950 μl assay buffer (1:20). This solution was used as the first point for the standard curve. For each plate, 0.5 ml was moved to 0.5 ml assay buffer, and did a 1:2 serial dilution. The 10 points are of the standard curve were: 25, 12.5, 6.25, 3.125, 1.56, 0.78, 0.39, 0.195, 0.098, and 0.049 ng/well. Each plate was washed again and serum samples were diluted at 1:10 in assay buffer. Standard curve and samples were loaded in 100 μl/well triplicates onto a 96-well plate, and incubated 3 h to detect total ntPE protein in serum samples. Each 96-well plate was then washed four times with wash buffer, and added 100 μl/well of rabbit anti-ntPE polyclonal antibody at 1:4000 dilutions and incubated for 2 h. Each 96-well plate was then washed four times with wash buffer, and added 100 μl/well of horseradish peroxidase (HRP) conjugated goat anti-rabbit IgG (Pierce, Cat. No.31460) at 1:4000 dilutions and incubated for 1 h. All incubation and coating steps were performed at room temperature on a shaker at 6 RPM. The HRP substrate, TMB (3,3′,5,5′tetramethylbenzidine), used to quantify bound antibody, was measured at 450 nm.

ELISA results are reported as the averages of the triplicate OD (450 nm) value of each sample. Concentrations were determined by the exceeding mean value plus three times the standard error of the mean (SEM) of the appropriate control value.

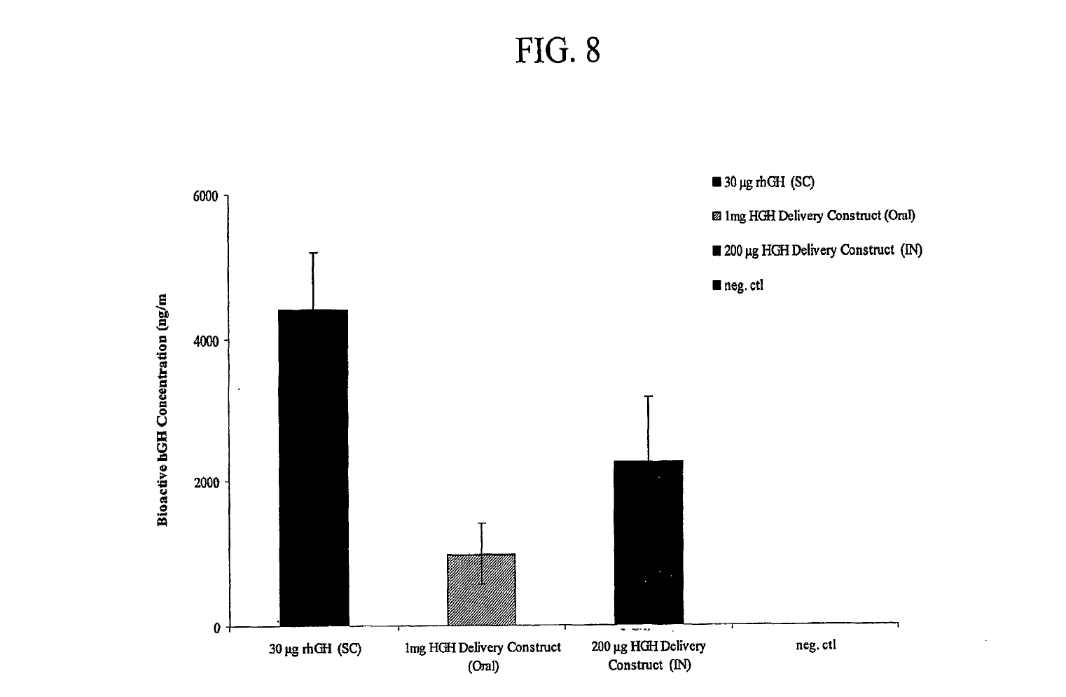

The results of the ELISA assays are presented in FIGS. 7-15. FIG. 7 shows serum concentrations of hGH 30 minutes following administration of either 30 μg rhGH SC, 300 μg hGH orally with HGH Delivery Construct, or 60 μg hGH with HGH delivery construct. FIG. 8 shows the amounts of bioactive hGH observed in the serum of mice administered 30 μg hGH subcutaneously, 300 μg hGH orally with the HGH Delivery Construct, and 60 μg hGH intranasally with the HGH Delivery Construct.

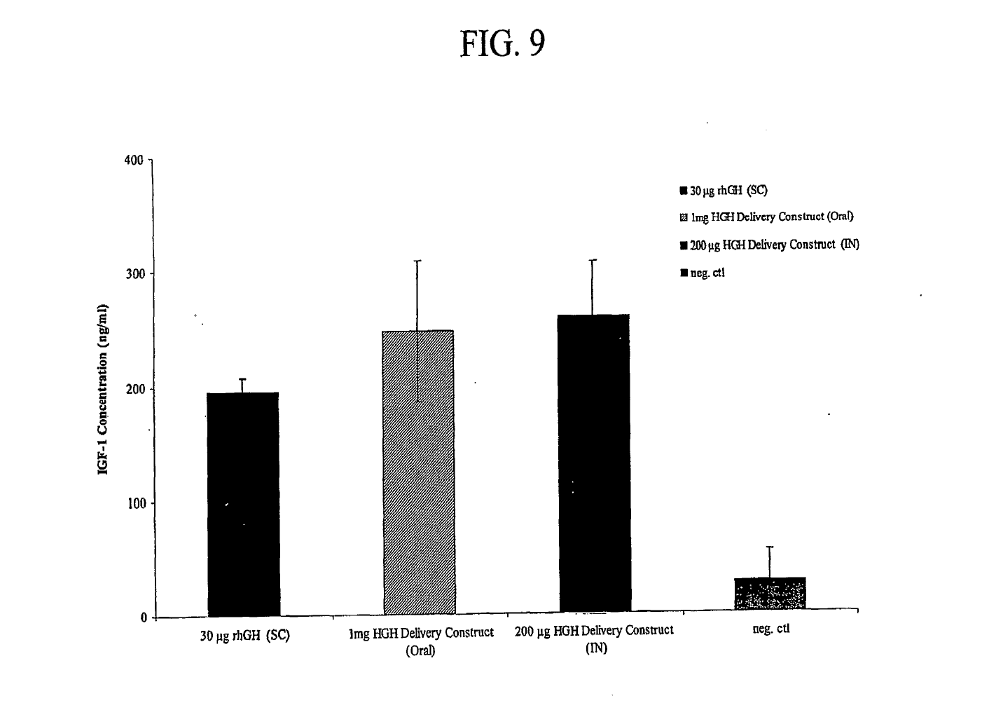

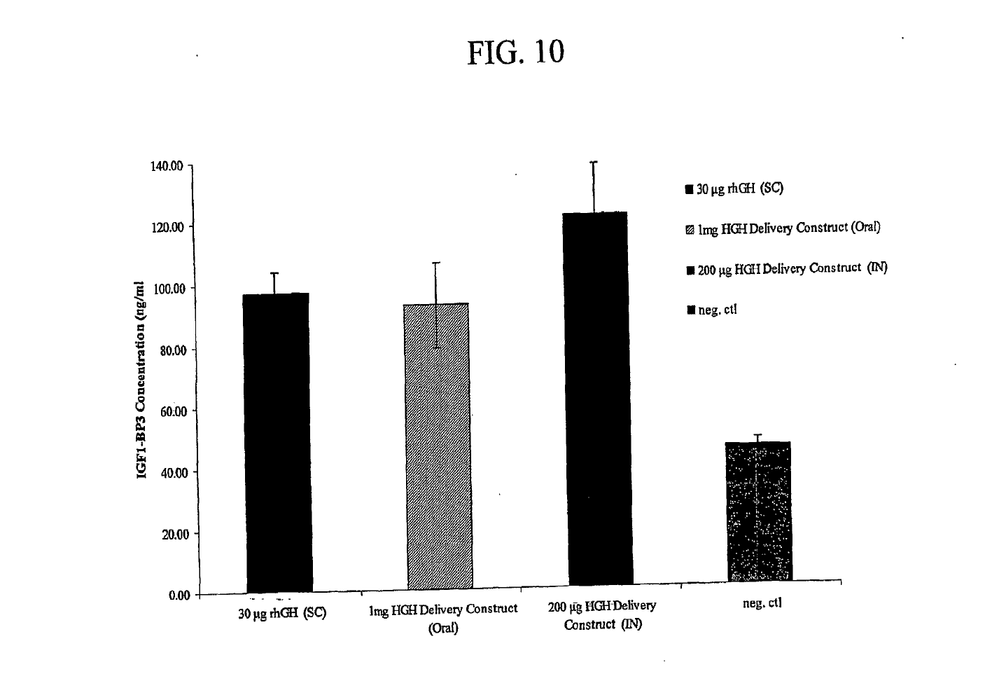

FIG. 9 shows the amounts of IGF-1 observed in the serum of mice administered 30 μg hGH subcutaneously, 300 μg hGH orally with the HGH Delivery Construct, and 60 μg hGH intranasally with the HGH Delivery Construct. FIG. 10 shows the amounts of IGF1-BP3 observed in the serum of mice administered 30 μg hGH subcutaneously, 300 μg hGH orally with the HGH Delivery Construct, and 60 μg hGH intranasally with the HGH Delivery Construct.

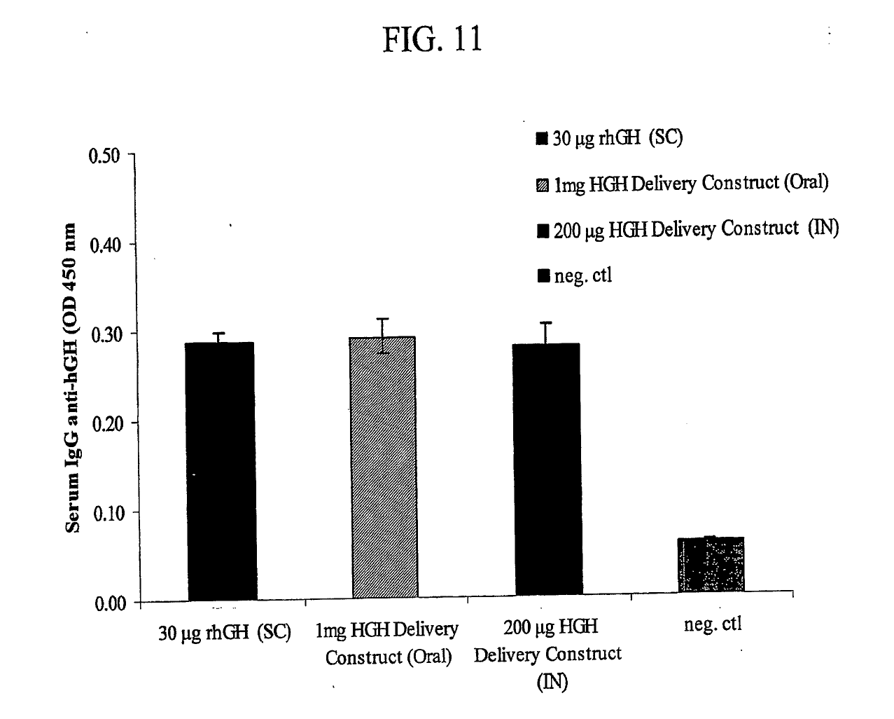

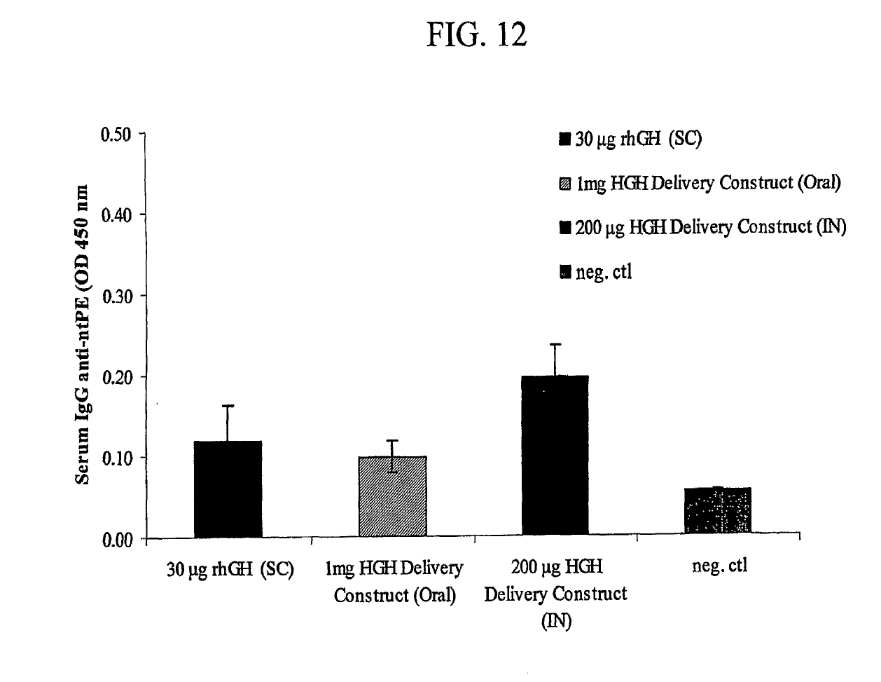

FIG. 11 shows the amounts of anti-hGH IgG antibodies observed in the serum of mice administered 30 μg hGH subcutaneously, 300 μg hGH orally with the HGH Delivery Construct, and 60 μg hGH intranasally with the HGH Delivery Construct. FIG. 12 shows the amounts of anti-ntPE IgG antibodies observed in the serum of mice administered 30 μg hGH subcutaneously, 300 μg hGH orally with the HGH Delivery Construct, and 60 μg hGH intranasally with the HGH Delivery Construct.

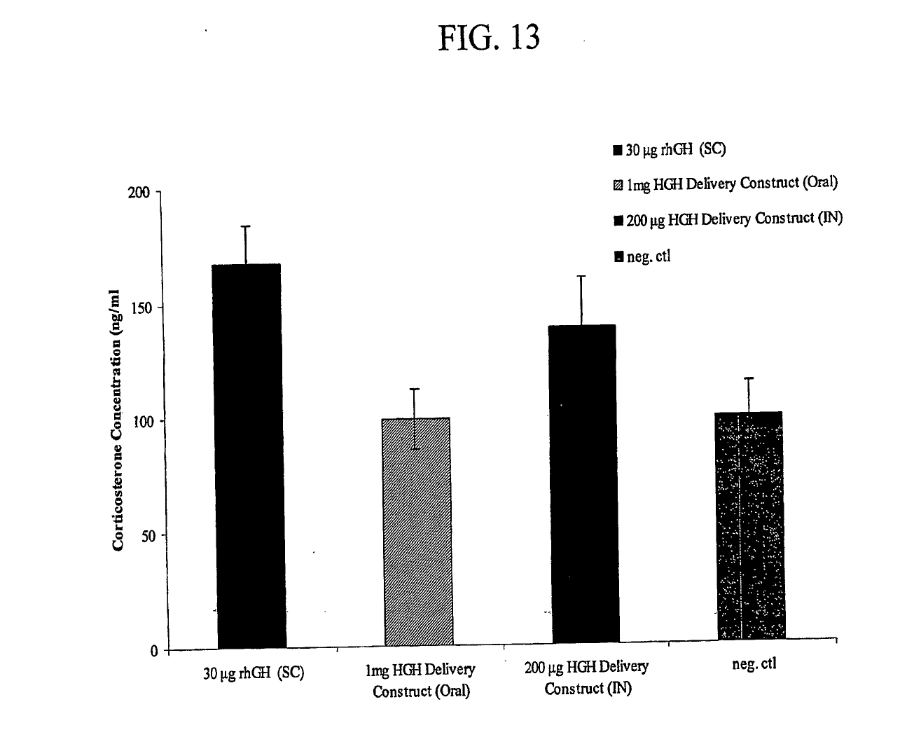

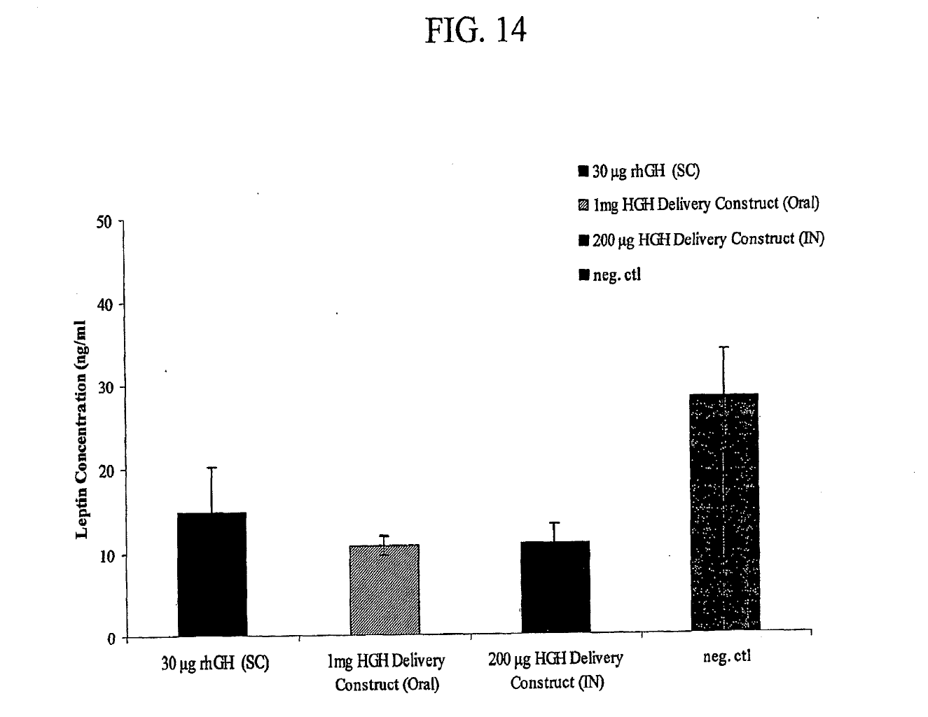

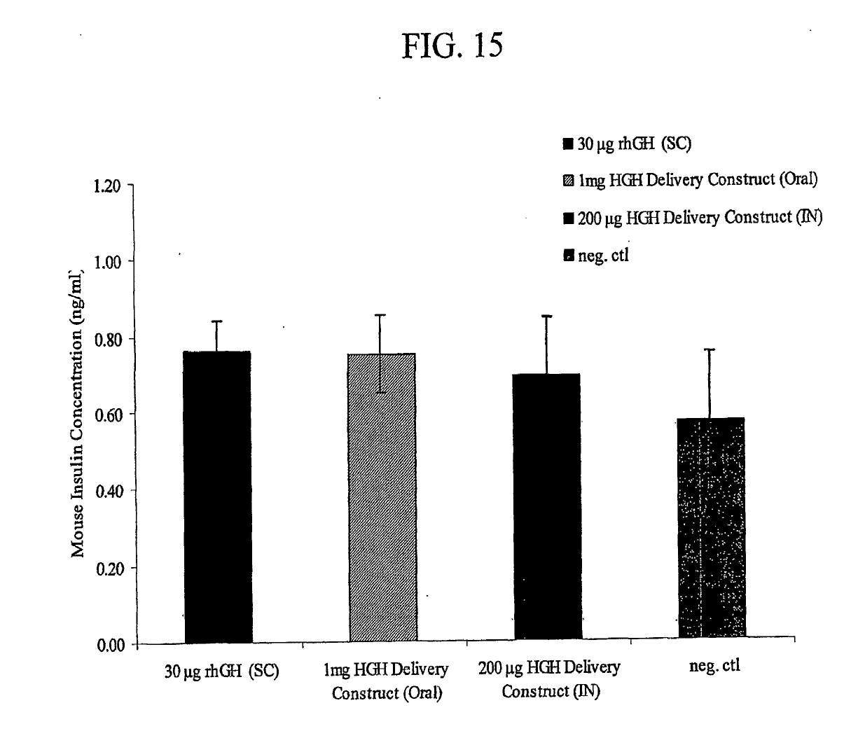

FIG. 13 shows amounts of corticosterone observed in the serum of mice administered 30 μg hGH subcutaneously, 300 μg hGH orally with the HGH Delivery Construct, and 60 μg hGH intranasally with the HGH Delivery Construct. FIG. 14 shows the amounts of leptin observed in the serum of mice administered 30 μg hGH subcutaneously, 300 μg hGH orally with the HGH Delivery Construct, and 60 μg hGH intranasally with the HGH Delivery Construct. FIG. 15 shows the amounts of insulin observed in the serum of mice administered 30 μg hGH subcutaneously, 300 μg hGH orally with the HGH Delivery Construct, and 60 μg hGH intranasally with the HGH Delivery Construct.

Taken together, FIGS. 7-15 show that both oral and intranasal administration of the HGH delivery construct are able to exert downstream effects similar to those caused by subcutaneous administration of hGH, especially relative to the negative control. Interestingly, administration of rhGH subcutaneously appears to result in a larger serum concentration of bioactive hGH relative to administration orally or intranasally with HGH Delivery Construct (FIG. 8), but downstream effects (e.g., IGF-1 and IGF1-BP3 expression) are comparable for the three routes of administration (FIGS. 9 and 10).

Mouse anti-hGH IgG antibody induction was comparable for each route of administration, though it should be noted that much more hGH (300 μg oral, 60 μg intranasal) was administered with the HGH Delivery Construct relative to the subcutaneous administration (30 μg) (FIG. 11). Oral administration of the HGH Delivery Construct appeared to induce a lower titer of antibodies against the ntPE portion of the delivery construct relative to intranasal administration. (FIG. 12).

6.6. Pharmacokinetics of Intranasal Administration of hGH in BALB/C Mice

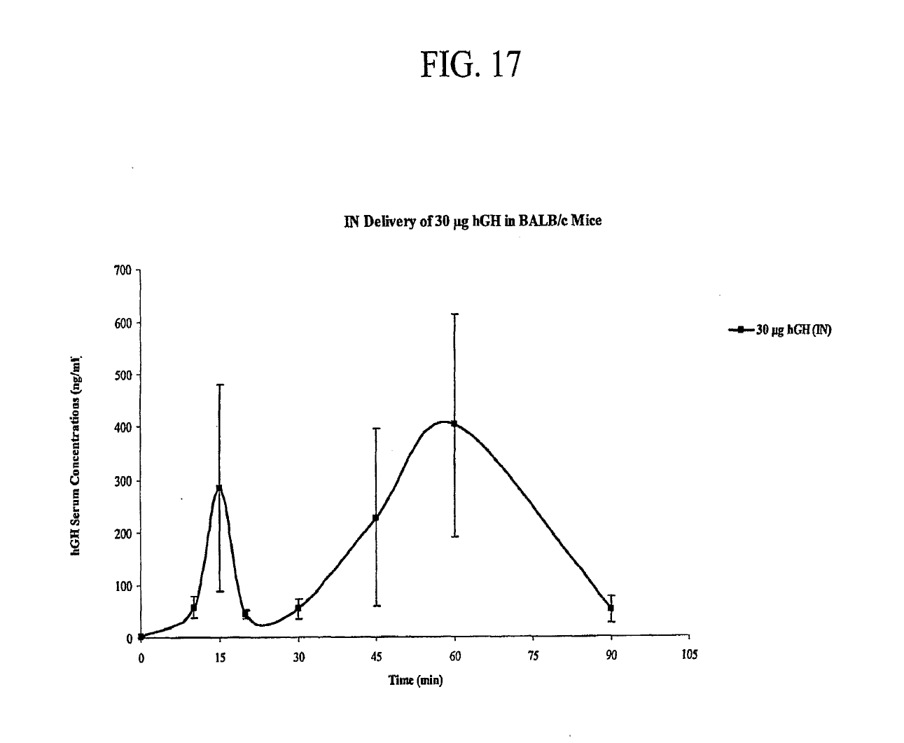

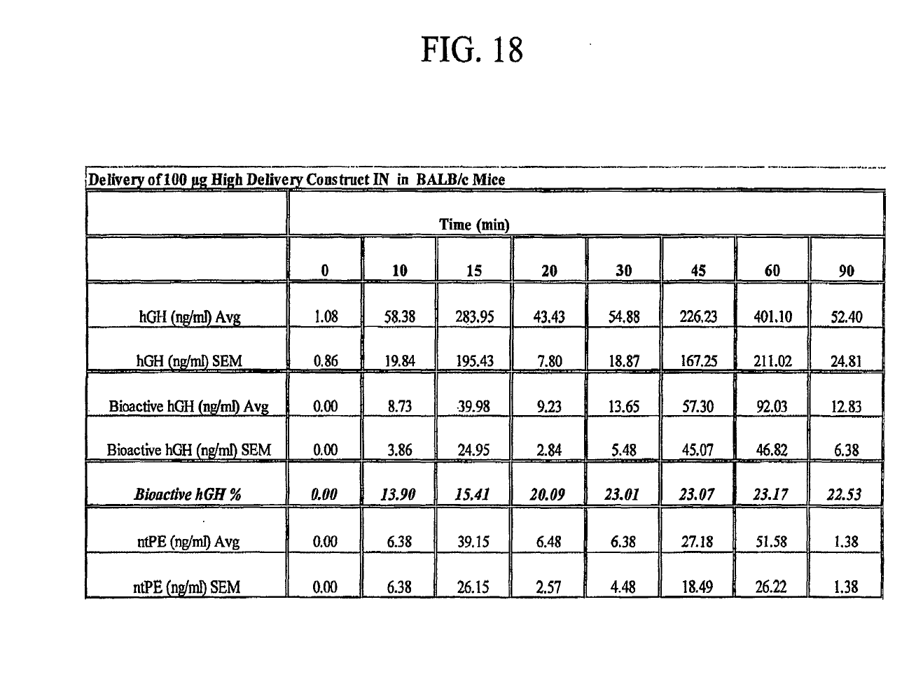

In this example, the pharmacokinetics of intranasal administration of 30 μg hGH with HGH Delivery Construct were monitored as follows. Four BALB/C mice per time point were intranasally administered an amount of HGH Delivery Construct corresponding to 30 μg hGH. Mice were sacrificed by CO₂ asphyxiation, exsanguinated, and the hGH, bioactive hGH, and ntPE serum concentrations determined by ELISA assay as described above. The results of the analysis are presented in tabular format as FIG. 18. The pharmacokinetic profile of hGH serum concentration is presented in graphical format as FIG. 17.

As shown in FIG. 17, peak serum concentrations of hGH were achieved 60 minutes following intranasal administration.

All publications and patent documents cited in this application are incorporated by reference in their entirety for all purposes to the same extent as if each individual publication or patent document were so individually denoted. Citation of these documents is not an admission that any particular reference is “prior art” to this invention.

What is claimed is:

1. A method for increasing the size of a subject by at least about 12%, comprising contacting an apical surface of a polarized epithelial cell of the subject with an amount of a delivery construct effective to increase the size of the subject by at least about 12%, wherein said delivery construct comprises a receptor binding domain, a transcytosis domain, a cleavable linker, and growth hormone (GH), wherein the transcytosis domain transcytoses the GH to and through the basal-lateral membrane of said epithelial cell, and wherein cleavage at said cleavable linker separates said GH from the remainder of said construct, thereby delivering the GH to the subject in an amount effective to increase the size of the subject by at least about 12%.

2. The method of claim 1, wherein said receptor binding domain is selected from the group consisting of receptor binding domains from Pseudomonas exotoxin A, cholera toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8.

3. The method of claim 1, wherein said receptor binding domain binds to a cell surface receptor selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

4. The method of claim 1, wherein said transcytosis domain is selected from the group consisting of transcytosis domains from Pseudomonas exotoxin A, botulinum toxin, diptheria toxin, pertussis toxin, cholera toxin, heat-labile E. coli enterotoxin, shiga toxin, and shiga-like toxin.

5. The method of claim 1, wherein said cleavable linker is cleavable by an enzyme that is selected from the group consisting of Cathepsin GI, Chymotrypsin I, Elastase I, Subtilisin AI, Subtilisin AII, Thrombin I, and Urokinase I.

6. The method of claim 1, wherein said cleavable linker comprises an amino acid sequence that is selected from the group consisting of Ala-Ala-Pro-Phe (SEQ ID NO.:1), Gly-Gly-Phe (SEQ ID NO.:2), Ala-Ala-Pro-Val (SEQ ID NO.:3), Gly-Gly-Leu (SEQ ID NO.:4), Ala-Ala-Leu (SEQ ID NO.:5), Phe-Val-Arg (SEQ ID NO.:6), Val-Gly-Arg (SEQ ID NO.:7).

7. The method of claim 1, wherein the epithelial cell is selected from the group consisting of nasal epithelial cells, oral epithelial cells, intestinal epithelial cells, rectal epithelial cells, vaginal epithelial cells, and pulmonary epithelial cells.

8. The method of claim 1, wherein the epithelial cell is a nasal epithelial cell.

9. The method of claim 1, wherein the epithelial cell is an intestinal epithelial cell.

10. The method of claim 1, wherein said subject is a human.

11. The method of claim 1, wherein said delivery construct contacts the apical membrane of the epithelial cell.

12. The method of claim 1, wherein said size of said subject is increased by at least about 13%.

13. The method of claim 1, wherein said size of said subject is increased by at least about 14%.

14. The method of claim 1, wherein said size of said subject is increased by at least about 15%.

15. The method of claim 1, wherein said size of said subject is increased by at least about 16%.

16. The method of claim 1, wherein said size of said subject is increased by at least about 17%.

17. The method of claim 1, wherein said size of said subject is increased by at least about 18%.

18. The method of claim 1, wherein said size of said subject is a weight of said subject.

19. The method of claim 1, wherein said size of said subject is a length of said subject.

20. The method of claim 1, wherein said size of said subject is a height of said subject.

21. The method of claim 1, wherein said GH is human growth hormone (hGH).

22. The method of claim 21, wherein said hGH has an amino acid sequence that is SEQ ID NO.:8.

23. The method of claim 1, further comprising performing the method of claim 1 a second time about 1 day after the method of claim 1 is performed the first time.

24. The method of claim 1, further comprising performing the method of claim 1 a second time about 2 days after the method of claim 1 is performed the first time.

25. The method of claim 1, further comprising performing the method of claim 1 a second time about 3 days after the method of claim 1 is performed the first time.


SEQUENCE LISTING (26 sequences)

SEQ ID NO: 1 - protein, 4 aa, Pseudomonas aeruginosa
  Cleavable linker
AAPF

SEQ ID NO: 2 - protein, 3 aa, Pseudomonas aeruginosa
GGF

SEQ ID NO: 3 - protein, 4 aa, Pseudomonas aeruginosa
AAPV

SEQ ID NO: 4 - protein, 3 aa, Pseudomonas aeruginosa
GGL

SEQ ID NO: 5 - protein, 3 aa, Pseudomonas aeruginosa
AAL

SEQ ID NO: 6 - protein, 3 aa, Pseudomonas aeruginosa
FVR

SEQ ID NO: 7 - protein, 3 aa, Pseudomonas aeruginosa
VGR

SEQ ID NO: 8 - protein, 192 aa, Homo sapiens
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 9 - protein, 266 aa, Pseudomonas aeruginosa
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKP

SEQ ID NO: 10 - protein, 153 aa, Pseudomonas aeruginosa
TVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNY

SEQ ID NO: 11 - protein, 5 aa, Pseudomonas aeruginosa
  VARIANT 5: Xaa = Any Amino Acid
YVADX

SEQ ID NO: 12 - protein, 5 aa, Pseudomonas aeruginosa
  VARIANT 2, 3, 5: Xaa = Any Amino Acid
DXXDX

SEQ ID NO: 13 - protein, 9 aa, Pseudomonas aeruginosa
  VARIANT 2: Xaa = Any Amino Acid
RXXXXXXRX

SEQ ID NO: 14 - protein, 9 aa, Pseudomonas aeruginosa
  VARIANT 2: Xaa = Any Amino Acid
KXXXXXXRX

SEQ ID NO: 15 - protein, 6 aa, Pseudomonas aeruginosa
  VARIANT 6: Xaa = Any Amino Acid
GRTKRX

SEQ ID NO: 16 - protein, 5 aa, Pseudomonas aeruginosa
  VARIANT 5: Xaa = Any Amino Acid
RVRRX

SEQ ID NO: 17 - protein, 6 aa, Pseudomonas aeruginosa
  VARIANT 6: Xaa = Any Amino Acid
DRVRRX

SEQ ID NO: 18 - protein, 6 aa, Pseudomonas aeruginosa
  VARIANT 2, 6: Xaa = Any Amino Acid
PXWVPX

SEQ ID NO: 19 - protein, 4 aa, Pseudomonas aeruginosa
  VARIANT 4: Xaa = Any Amino Acid
WVAX

SEQ ID NO: 20 - protein, 4 aa, Pseudomonas aeruginosa
  VARIANT 1, 3, 4: Xaa = Any Amino Acid
XFXX

SEQ ID NO: 21 - protein, 4 aa, Pseudomonas aeruginosa
  VARIANT 1, 3, 4: Xaa = Any Amino Acid
XYXX

SEQ ID NO: 22 - protein, 4 aa, Pseudomonas aeruginosa
  VARIANT 1, 3, 4: Xaa = Any Amino Acid
XWXX

SEQ ID NO: 23 - protein, 10 aa, Pseudomonas aeruginosa
  VARIANT 9, 10: Xaa = Any Amino Acid
DRYIPFHLXX

SEQ ID NO: 24 - DNA, 1839 bp, Pseudomonas aeruginosa
gccgaagaagctttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaag

SEQ ID NO: 25 - DNA, 37 bp, Pseudomonas aeruginosa
aactgcagggaggcttacgccagcctcgactgcagaa

SEQ ID NO: 26 - DNA, 37 bp, Pseudomonas aeruginosa
ttctgcagtcgaggctggcgtaagcctccctgcagtt